(19) United States
(12) Patent Application Publication
(10) Pub. No.: US 2026/0144817 A1
(43) Pub. Date: May 28, 2026

(54) INTRAVESICAL TIL THERAPY IN BCG-EXPOSED PATIENTS

(71) Applicant: H. LEE MOFFITT CANCER CENTER AND RESEARCH INSTITUTE, INC., Tampa, FL (US)

(72) Inventors: Michael POCH, Tampa, FL (US); Katarzyna A. REJNIAK, Tampa, FL (US); Shari PILON-THOMAS, Land O Lakes, FL (US)

(21) Appl. No.: 19/122,032

(22) PCT Filed: Oct. 18, 2023
(86) PCT No.: PCT/US2023/077204

§ 371 Date: Apr. 17, 2025

Related U.S. Application Data
(60) Provisional application No. 63/417,186, filed on Oct. 18, 2022.

(51) Int. Cl. A61K 35/17; A61K 31/7068; A61K 40/11; A61K 40/15; A61K 40/17; A61K 40/31; A61P 35/00

(52) U.S. Cl. CPC A61K 35/17; A61K 31/7068; A61K 40/11; A61K 40/15; A61K 40/17; A61K 40/31; A61P 35/00

(57) ABSTRACT

Disclosed are compositions and methods for the treatment of cancer through administration of gemcitabine, tumor infiltrating lymphocytes and bacillus Calmette-Buerin (BCG) as well as models for predicting responsiveness to said treatment.

### II. CROSS REFERENCE TO RELATED APPLICATIONS

This application claims the benefit of U.S. Provisional Application No. 63/417,186, filed on Oct. 18, 2022, which is incorporated herein by reference in its entirety.

### I. STATEMENT OF GOVERNMENT SUPPORT

This invention was made with government support under Grant No. CA259387 awarded by National Institutes of Health. The government has certain rights in the invention.

### III. BACKGROUND

Bladder Cancer is the fourth most common cancer in men and a leading cause of cancer death among men and women. There will be approximately 86,000 new cases of bladder cancer diagnosed in 2021 and approximately 17,000 bladder cancer deaths in the United States. There are nearly 500,000 people living with bladder cancer in the United States. Despite current therapies, 50% of patients with intermediate and high risk localized disease fail bladder sparing treatment. This is particularly meaningful given that recurrent and/or locally advanced tumors have a worse cancer specific prognosis often requiring radical cystectomy, a potentially high risk and quality of life changing operation. In addition, approximately 25% of patients present with advanced stage disease. Newer therapies and clinical trial results have unfortunately still yielded efficacy of less than 50%. This is particularly impactful for US veterans for whom bladder cancer is also the fourth most common cancer. In addition smoking, a known risk factor for bladder cancer, has a higher incidence of higher grade bladder cancers at diagnosis. This is also significant for the veteran population who have a higher incidence of smoking. There is a clear need for a novel approach to treat this disease at all stages of disease.

### IV. SUMMARY

Disclosed are methods and compositions related to treatment of a cancer in a BCG unresponsive subject.

Disclosed herein are methods of treating, inhibiting, reducing, decreasing, ameliorating, and/or preventing a cancer and/or metastasis (such as, for example, bladder cancer including, but not limited to localized non-muscle invasive bladder cancer) in a Bacillus Calmette-Guerin (BCG) unresponsive subject comprising administering to the subject an adoptive cell therapy (ACT) (such as, for example, administration (including intravesical administration) of an immune cell selected from the group consisting of tumor infiltrating lymphocytes (TILs), marrow infiltrating lymphocytes (MILs), chimeric antigen receptor (CAR) T cells, CAR macrophage (CARMA), CAR Natural Killer cells (CAR NK cells), and CAR NK T cells).

Also disclosed herein are methods of treating, inhibiting, reducing, decreasing, ameliorating, and/or preventing a cancer and/or metastasis of any preceding aspect, wherein the treatment further comprises the administration of BCG.

In one aspect, disclosed herein are methods of treating, inhibiting, reducing, decreasing, ameliorating, and/or preventing a cancer and/or metastasis of any preceding aspect, further comprising administering to the subject gemcitabine. In some aspects, the immune cells are administered before, concurrent with, simultaneously, or after administration of gemcitabine.

Also disclosed herein are methods of treating, inhibiting, reducing, decreasing, ameliorating, and/or preventing a cancer and/or metastasis of any preceding aspect, wherein the method does not require preconditioning of the TILs prior to administration.

In one aspect, disclosed herein are methods of treating, inhibiting, reducing, decreasing, ameliorating, and/or preventing a cancer and/or metastasis of any preceding aspect, wherein the immune cells are expanded ex vivo prior to administration. In one aspect, the method further comprises selecting tumor-reactive immune cells after ex vivo expansion.

### V. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1. Immune infiltrates in bladder tumors. Primary bladder tumors were digested to single cells and the percentage of immune infiltrates were measured by flow cytometry.

FIG. 2: TIL expansion from bladder tumors. The total number of TIL expanded from bladder or LN tumor fragments was measured at 4 weeks after initiation of culture. Each point represents the total TIL generated from each fragment within an individual patient.

FIG. 3: Phenotype of TIL Expanded from Primary Bladder Tumors. At four weeks after the initiation of TIL cultures, TIL were collected from each fragment and the percentage of CD8⁺ T cells, CD3⁺ T cells, CD4⁺ T cells, CD8⁺ T and CD3⁻CD56⁺ NK cells were measured by flow cytometry. Each point represents the mean percentage of cells generated from each fragment for an individual patient.

FIG. 4: Bladder TIL is specific for autologous tumor. Expanded TIL was collected from individual fragments at 4 weeks after initiation of culture. TIL were co-cultured in complete media alone (CM), or at a 1:1 ratio with digested autologous tumor cells for 24 hours and supernatants were collected. IFN-gamma in supernatants was measured by ELISA.

FIG. 5: Neoantigen-specificity of TIL. TIL reactivity to specific neoantigen peptides expressed in autologous tumor. T2 cells were pulsed with peptide pools or individual peptides and co-cultured with autologous TIL. IFN-gamma production was measured by ELISPOT.

FIG. 6 Expansion of TIL from bladder tumors stratified by BCG immunotherapy. The total number of TIL expanded from bladder or LN tumor fragments was measured at 4 weeks after initiation of culture. A) TIL growth stratified by previous BCG immunotherapy and median number of expanded TIL in 37 patients.

FIG. 7 Phenotype of TIL expanded from primary bladder tumors previously treated with BCG immunotherapy. Eight BCG-treated patients were matched with 8 BCG-untreated patients measured at 4 weeks after initiation of culture

FIG. 8: Suppressive MDSC are present in the urine of bladder cancer patients. (A) Immune subsets were measured in urine collected from an individual bladder patient. (B) MDSC were purified from urine using an AutoMACs separation kit. T cells stimulated with OKT3 antibody alone or co-cultured with MDSC at various ratios. After 24 hours, supernatants were collected and IFN-gamma was measured by ELISA.

FIGS. 9A-C Detection of intravesically transferred T cells. (A) Ultrasound of a tumor growing in the bladder of a mouse. (B) CTV+ OT-I T cells infused intravesically are detected within bladder tumor by flow cytometry (far right). (C) Tumor regression induced by intra-vesical transfer of tumor-reactive T cells.

FIGS. 10A-C: MB49 TIL and efficacy of intravesical delivery of TIL. MB49 tumors (n=4) were resected and digested to single cell suspensions. (A) Expression of checkpoint proteins on CD8+ T cells was measured by flow cytometry. (B) Reactivity of TIL against irrelevant and relevant tumor targets. (C) Efficacy of inV delivery of TIL in mice bearing MB49 bladder tumors. CTL=control, untreated mice; TIL=mice treated with inV TIL

FIG. 11: Schematic of a Virtual Clinical Trial predictor. Uses patient histology, image analysis and computer simulations for the Learning phase, and predicts tumor chemoresistance at the Translational phase.

FIGS. 12A-K. In silico model of in vivo tumors based on in vitro data (a,b) and histology (c), with simulated metabolic gradients (e), tumor growth (f,i), and its composition (g-k). Bottom: equations.

FIG. 13. Multilevel classification of a set of 38 clinical data. The tree binary endpoints indicate successful TIL expansion (filled circles) or no TIL expansion (open circles).

FIG. 14A macroscopic model of tumor-T cell-vaccine-anti-PD1 inter-actions fitted to experimental data (black dots with +/−SEM); tumor growth lines: control (blue), vaccine treatment (green), vaccine+PD-1 inhibitor treatment (red). Inset shows the interaction flowchart.

FIG. 15 MADS performance chart for a three-drug combination (HAP-Vaso-Sens) optimized to maximize dead cell number. Shown: the examined cases (dots), optimal solution (star), method convergence (inset)

FIG. 16A-C show MB49 orthotopic tumors (n=3) were resected and digested to single cell suspensions. TIL were isolated and plated in IL-2. (A) The number of wells increased from 1 to 24 in 20 days. (B) TIL were tested for reactivity against MB49 tumor cells by co-culture and IFN-gamma ELISA. (C) The majority of cells were CD3+ CD8+ T cells (upper left quadrant) as determined by flow cytometry.

FIGS. 17A-C (A) CD8+ T cell infiltration per mg of tumor; (B-C) IHC images of control (B) and emm55-treated (C) tumor.

FIG. 18 Tumor regression induced by DC vaccine, chemo, and combination of both.

FIG. 19 Schematics of the hierarchical model design of combination treatment: (i) tumor cells (C_{U}, C_{T}=0), (ii) T cells (T_{A} & T_{E}, dead tumor cells C_{D}), (iii) T+vaccine (V, C_{U}, C_{T}), (iv) T+anti-PD1 (A), (v) T+dendritic vaccine (DC), (vi) C+T+V+A+DC. Feedback: positive (proliferation, activation, recruitment): solid lines, negative (suppression, killing): dashed lines: influx/outflux: dotted lines.

FIG. 20 Graphical User Interface (GUI) for Organoid3D model: the simulated organoid fitted to in vitro data.

FIG. 21 Production of IFN-gamma by T cells exposed in vitro to various levels of O₂ and pH.

FIG. 22 T cells were cultured for 72 hrs at normal oxygen levels (normoxia) or at 1% oxygen (hypoxia) with anti-CD3 stimulation. 72 hours post-activation, the expression of memory markers on CD8+ T cells was measured.

FIGS. 23A and B A. Effect of in vitro checkpoint targeting on TIL expansion; B. Expression of co-inhibitory and co-stimulatory receptors

FIGS. 24A-D Mathematical agent-based model of TIL infiltration and cytokine pattern in tumor microenvironment. (A) histology sample for (B) tissue digitization and (C) oxygen gradient simulation within the tissue. (D) IFN-gamma secretion and diffusion (inset), and individual tumor cell exposure (histogram).

FIG. 25 PDX tumor growth measured in ACT-treated in control (No ACT) mice.

FIG. 26 shows schematics of the combination therapy model: (I) tumor cells (C), (II) TIL (T), (III) TIL+anti-PD1 (A), (IV) TIL+DC vaccine (DV), (V) TIL+DV+Gemcitabine (Gem). Solid lines: positive feedback (proliferation, activation, recruitment), dashed lines: negative feedback (suppression, killing), dotted lines: influx/outflux.

FIG. 27 shows (left) an example of stable oxygen distribution, (right) the average oxygen steadily increases and reaches equilibrium at ˜22 mmHg.

FIGS. 28A, 28B, 28C, and 28D show in vivo and in silico work. FIG. 28A shows immunocompetent mice were injected with MB-OVA tumor cells then treated with gemcitabine and/or adoptive cell therapy with OT1 cells. FIG. 28B shows that resected tumors were sliced and stained for cells (H&E), vasculature (CD31) and immune cells (CD4+, CD8+, CD11b, Ly6G), then scanned and digitized. FIG. 28C shows that repulsive were calculated for cell-to-cell and cell-to-vessel interactions to avoid overlapping. FIG. 28D shows the 12 digitized tissues. The black lines inside some tissues enclose the tumor regions.

FIGS. 29A, 29B, and 29C show analysis of cells' oxygenation and clustering patterns in the tumor regions. FIG. 29A shows in panel (i) A portion of GEM tissue showing the immune cells in different oxygenated regions. FIG. 29A in panel (ii) shows histograms of the oxygenation level and minimum distances of CD8+ cells from the closest vessel. FIG. 29B shows the Ripley's K analysis of CD8+ and MDSCs cells across the treatments (solid line above—clumped, below dispersed). FIG. 29C shows in panel (i) empirical cumulative distribution functions for different (left) and similar (right) distributions. FIG. 29C in panel (ii) shows Kolgomorov-Smirvov pairwise comparison. FIG. 29C in panel (iii) shows Kruskal-Wallis p-values for comparing distributions across the treatments grouped by tumor sizes (black-similar, red-different) for the well-oxygenated and hypoxic cells.

FIGS. 30A, 30B, and 30C show simulated oxygen landscapes. FIG. 30A shows a model of the vascular influx as a boundary condition. FIG. 30A in panel (i) shows the pO₂ in a vessel is constant. FIG. 30A in panel (ii) the pO₂ at each grid point surrounding the vessel is inversely proportional to the distance from the vessel center. FIG. 30B shows the cellular uptake is modelled in a similar manner. FIG. 30C shows the numerically stable oxygen maps. The smaller tissues (first and second rows) are well oxygenated and have fewer hypoxic regions compared to the larger tissues (last row).

FIGS. 31A and 31B show the immune landscape in bladder cancer after treatment. FIG. 31A shows histology from mouse bladder tumors: untreated, and treated with gemcitabine (GEM), adoptive T cell therapy (OT-I), and combination of GEM and OT-I, were segmented into tumor and nontumor regions (top row), digitized into immune and tumor cells (middle row), and used to simulate the stable oxygen distribution (bottom row). FIG. 31B shows the immune cell proportions varied between the large (Lrg) and small (SmA and SmB) bladder tissues, and across tumor and nontumor regions. The histograms and empirical distributions of oxygenated CD8+, CD11b+, Ly6G+, and all cells showed different distributions for untreated tumor and the treated tumors.

FIGS. 32A, 32B, 32C, and 32D show the oxygen landscape of IPMN tumors. Pancreas tumors of different grades i) benign ii) premalignant with fibrotic stroma, iii) invasive with desmoplastic stroma, were discretized (32A) and used to simulate the stable oxygen distribution (32B). The hypoxic cells were identified (32C). FIG. 32D shows the histograms and empirical distributions of oxygenated vs. hypoxic tumor cells showed different distributions of: benign tissue-Normal (17.72,6.82), pre-malignant tumor-Gamma (0.71, 0.11), and invasive tumor-Gamma (0.4,0.072).

### VI. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant. 47. A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. METHOD OF TREATING CANCER

### 1. Immunotherapy and Bladder Cancer.

Bladder cancer has long been recognized as a malignancy that is responsive to immune-based therapy. As early as the 1970s, patients with localized non-muscle invasive bladder cancer (NMIBC) have been treated with intravesical installations of Bacillus Calmette-Guerin (BCG), a live attenuated strain of Mycobacterium bovis. Induction intravesical BCG, considered standard of care for high risk non-muscle invasive disease, has multiple mechanisms of action for anti-tumor activity. These mechanisms include direct binding of tumor cells and initiation of a Th1 mediated immune response with CD4⁺ T cells and CD8⁺ cytotoxic T lymphocytes. In addition, stimulated by BCG, Tumor Necrosis Factor related apoptosis ligand (TRAIL) released by neutrophils has also been demonstrated to have anti-tumor effects in bladder cancer. Tumors characterized by high CD4⁺ T cells, low regulatory T cells (Tregs), and low CD68⁺ or CD163⁺ macrophages are associated with prolonged recurrence free survival in patients that respond to BCG therapy. In clinically advanced bladder cancer, targeting the PD-1/PD-L1 pathway has demonstrated an improvement in overall survival of 4-8 months with some durable responses but with only a 20% response rate overall. Unfortunately, this still leaves median overall survival (OS) slightly over one year in patients with metastatic disease and even worse for non-responders.

### 2. Adoptive Cell Therapy.

One major advance for the treatment of solid tumors has been the success of adoptive T cell therapy (ACT) where autologous tumor-infiltrating T lymphocytes (TIL) are expanded and activated ex vivo and then reinfused into the cancer patient. Indeed, TIL have emerged as one of the most powerful therapies for unresectable metastatic melanoma, with a 50% response rate. Our group has extensive experience expanding TIL from multiple tumor types and have applied ACT with TIL to achieve long-lasting responses in mouse models of cancer and in patients with incurable metastatic melanoma. The premise behind this approach is that tumors are enriched in tumor-specific T cells. In the tumor microenvironment, these TIL are functionally unresponsive but can become re-activated. ACT depends upon infiltration of T cells into tumors prior to harvest and ex vivo expansion of TIL. After surgical resection, tumors are minced into 3-5 mm² fragments and cultured in growth media containing interleukin-2 (IL-2). Each pool is expanded individually and then screened for tumor specific activity against autologous tumor cells. The initial expansion of the TIL is followed by the second rapid expansion phase (REP) to generate up to 150 billion or more cells. Patients undergo non-myeloablative (NMA) chemotherapy prior to infusion of TIL. This strategy has shown efficacy in several types of solid tumors, thus an adoptive cell therapy (ACT) with TIL has the potential to improve clinical outcomes in patients with bladder cancer. Previous ACT TIL therapies for melanoma, non-small cell lung cancer and sarcoma have been done in the mestastatic setting with systemic administration. This treatment requires a number of steps in order for the systemic administration of TIL to be effective. Prior to administration of TIL patients need to undergo myeloablation with a cytotoxic chemotherapy regimen that consists of cyclophosphamide and fludarabine. The toxicity of this regimen can be as high as 30-40%. After myeloablation, TIL is infused followed infusions of high dose IL-2. High dose IL-2 has been shown to have significant toxicity as high as 70-90% of which includes severe hypotension requiring vasopressors and intensive care admission. Translating ACT in bladder cancer provides a unique opportunity to deliver TIL intravesically by administration of T cells through a catheter into the bladder directly to tumors. Since this is a more localized treatment, it is anticipated that TIL can be injected more frequently, in lower quantities, and in the absence of systemic cytotoxic chemotherapy required for the induction of lymphodepletion and high dose IL-2 both of which are associated with significant toxicity.

### 3. TIL in Bladder Cancer.

Bladder tumors have a high mutational burden corresponding to an increased number of neoantigens. These mutations can lead to the expression of non-self, or “foreign” proteins, which can be recognized by activated T cells at the tumor site. TIL were first isolated from urological tumors in the early 1990s. The majority of the lymphocytes infiltrating the tumors were CD3⁺ T cells. In primary bladder tumors, the presence of CD8⁺ T cells correlated with lower stage disease. T cells within tumors demonstrated a cytotoxic but exhausted phenotype and T cell function can be rescued ex vivo. TIL expanded from bladder tumors demonstrate cytotoxic effects against autologous tumor. For patients with advanced disease, the presence of CD8⁺ TIL is associated with improved survival. Thus, while the profile of T cells in bladder cancer are predictive of clinical outcomes, the T cells are not able to suppress tumor growth. Strategies to improve infiltration, expansion, or activity of antigen-reactive T cells at the tumor site can lead to successful tumor regression in patients with bladder cancer.

### 4. Development of Novel Immunotherapies for Bladder Cancer.

In this proposal, we perform safety and efficacy phase I/II clinical trial of intravesical TIL therapy in BCG unresponsive patients. We can evaluate whether it is feasible and safe to deliver TIL into the bladder of BCG unresponsive patients. We can measure the specificity of expanded TIL and develop a computational tool to predict growth of effective TIL from a given tumor based on its histology and patient's demographics.

Disclosed herein are methods of treating, inhibiting, reducing, decreasing, ameliorating, and/or preventing a cancer and/or metastasis (such as, for example, bladder cancer including, but not limited to localized non-muscle invasive bladder cancer) in a Bacillus Calmette-Guerin (BCG) unresponsive subject comprising administering to the subject an adoptive cell therapy (ACT) (such as, for example, administration (including intravesical administration) of an immune cell selected from the group consisting of tumor infiltrating lymphocytes (TILs), marrow infiltrating lymphocytes (MILs), chimeric antigen receptor (CAR) T cells, CAR macrophage (CARMA), CAR Natural Killer cells (CAR NK cells), and CAR NK T cells).

Also disclosed herein are methods of treating, inhibiting, reducing, decreasing, ameliorating, and/or preventing a cancer and/or metastasis of any preceding aspect, wherein the treatment further comprises the administration of BCG.

In one aspect, disclosed herein are methods of treating, inhibiting, reducing, decreasing, ameliorating, and/or preventing a cancer and/or metastasis of any preceding aspect, further comprising administering to the subject gemcitabine. In some aspects, the immune cells are administered before, concurrent with, simultaneously, or after administration of gemcitabine.

Also disclosed herein are methods of treating, inhibiting, reducing, decreasing, ameliorating, and/or preventing a cancer and/or metastasis of any preceding aspect, wherein the method does not require preconditioning of the TILs prior to administration.

In one aspect, disclosed herein are methods of treating, inhibiting, reducing, decreasing, ameliorating, and/or preventing a cancer and/or metastasis of any preceding aspect, wherein the immune cells are expanded ex vivo prior to administration. In one aspect, the method further comprises selecting tumor-reactive immune cells after ex vivo expansion.

The disclosed compositions can be used to treat any disease where uncontrolled cellular proliferation occurs such as cancers. A representative but non-limiting list of cancers that the disclosed compositions can be used to treat is the following: lymphomas such as B cell lymphoma and T cell lymphoma; mycosis fungoides; Hodgkin's Disease; myeloid leukemia (including, but not limited to acute myeloid leukemia (AML) and/or chronic myeloid leukemia (CML)); bladder cancer (including, but not limited to localized non-muscle invasive bladder cancer); brain cancer; nervous system cancer; head and neck cancer; squamous cell carcinoma of head and neck; renal cancer; lung cancers such as small cell lung cancer, non-small cell lung carcinoma (NSCLC), lung squamous cell carcinoma (LUSC), and Lung Adenocarcinomas (LUAD); neuroblastoma/glioblastoma; ovarian cancer; pancreatic cancer; prostate cancer; skin cancer; hepatic cancer; melanoma; squamous cell carcinomas of the mouth, throat, larynx, and lung; cervical cancer; cervical carcinoma; breast cancer including, but not limited to triple negative breast cancer; genitourinary cancer; pulmonary cancer; esophageal carcinoma; head and neck carcinoma; large bowel cancer; hematopoietic cancers; testicular cancer; and colon and rectal cancers.

It is understood and herein contemplated that the disclosed treatment regimens can used alone or in combination with any anti-cancer therapy known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). The treatment methods can include or further include checkpoint inhibitors including, but are not limited to antibodies that block PD-1 (such as, for example, Nivolumab (BMS-936558 or MDX1106), pembrolizumab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep).

### 5. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and Mckenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A.R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### C. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

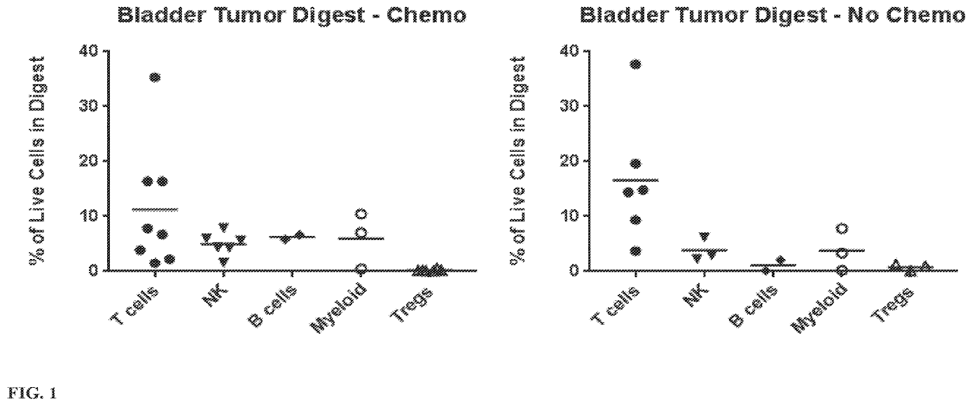

90. We first examined the infiltration of immune cells into primary tumors resected from bladder cancer patients. Primary bladder tumors obtained from radical cystectomy were mechanically and enzymatically digested using media containing 2% Collagenase Type IV and a GentleMACS Dissociator (Miltenyi). Flow cytometric analysis of immune subsets was performed. We evaluated the percentage of CD3⁺ T cells, CD3⁻CD56⁺ Natural Killer (NK) cells, CD19⁺ B cells, CD11b⁺ myeloid cells, and CD4⁺CD25⁺foxp3⁺ regulatory T cells (Tregs). As shown in FIG. 1, immune infiltrate patterns were similar in tumors resected from patients that were treatment naïve (no chemo) or treated with chemotherapy prior to surgery. These results support the infiltration of T cells into bladder tumors and suggest that pre-treatment with chemotherapy does not alter the immune cell composition within bladder tumors.

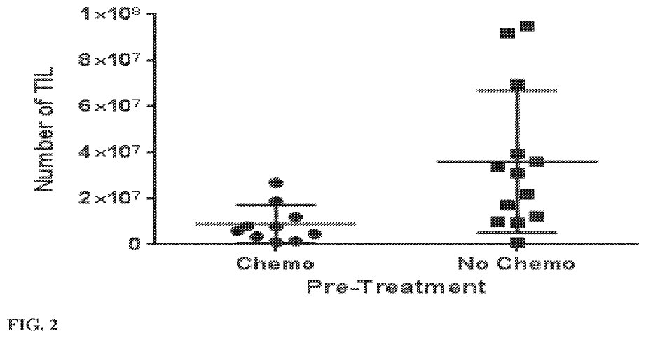

In an initial clinical study, tumors obtained from radical cystectomy specimens or resected lymph node (LN) metastases were collected from bladder cancer patients. The feasibility of TIL expansion was evaluated in 20 primary bladder tumor samples and 7 LN metastatic lesions. Thirteen samples (46%) were collected from patients who had received neoadjuvant chemotherapy (NAC). Tumors were minced into fragments, placed in individual wells of a 24-well plate, and propagated in media containing 6000 IU/mL IL-2 for four weeks. Of the bladder tumors collected, 70% of primary tumors demonstrated TIL expansion. When we compared between patients that were untreated or previously treated with chemotherapy prior to surgery, we found no significant difference in the ability to expand TIL (FIG. 2).

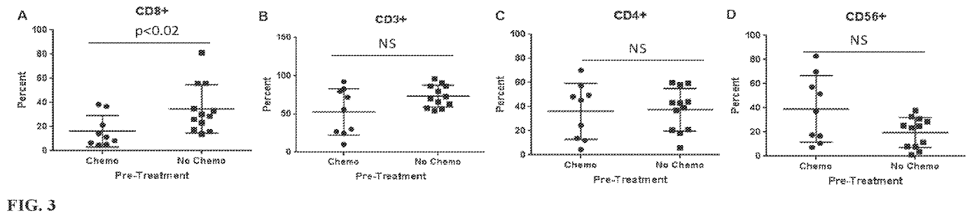

Expanded TIL were predominantly CD3⁺ with an increased percentage of CD8⁺ T cells expanded from tumors of patients that had not been treated with chemotherapy prior to surgery (FIG. 3A). No difference was measured in total CD3⁺ or CD4⁺ T cells or NK cells (FIG. 3B-D). Together, these studies demonstrate the feasibility of expanding TIL from the tumors of bladder cancer patients regardless of pre-treatment with chemotherapy.

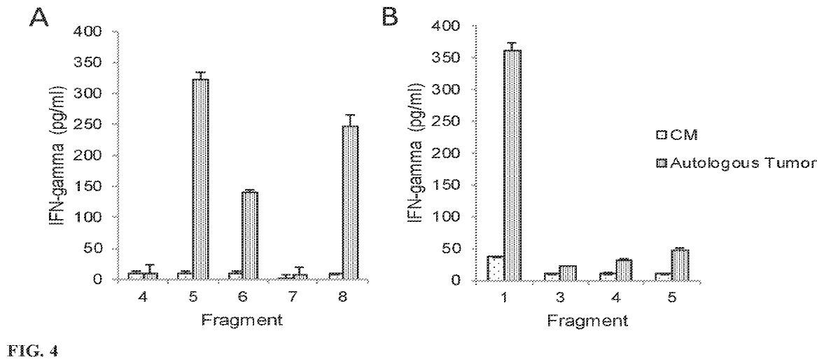

We next evaluated whether TIL grown from bladder tumor fragments demonstrated tumor-specific activity. Anti-tumor reactivity was assessed after co-culture of expanded TIL with autologous tumor digest and IFN-gamma production was measured by ELISA. TIL secreted IFN-gamma in response to autologous tumor in 50% of patients. Reactivity of TIL to autologous tumor for 2 individual patients is shown in FIG. 4. Similar to melanoma, not every fragment led to the expansion of tumor-reactive TIL but at least 1 fragment per patient demonstrated tumor-reactive TIL activity. This study was the practical first step towards an autologous TIL therapy process for therapeutic testing in patients with bladder cancer.

We next evaluated whether expanded TIL responded to specific neoantigens expressed by autologous tumor in an HLA-A2+ patient. Mutations in tumor were defined using whole exome and RNA sequencing. A peptide-binding algorithm was used to predict potential epitopes restricted to HLA-A2. A total of 48 peptides were predicted. An initial screen using pools of peptides was performed. As shown in FIG. 5, TIL responded to peptides in pools 4 and 5. Upon additional screening, 4 individual peptides were identified. This initial study demonstrates that TIL recognize neoantigens in bladder tumors.

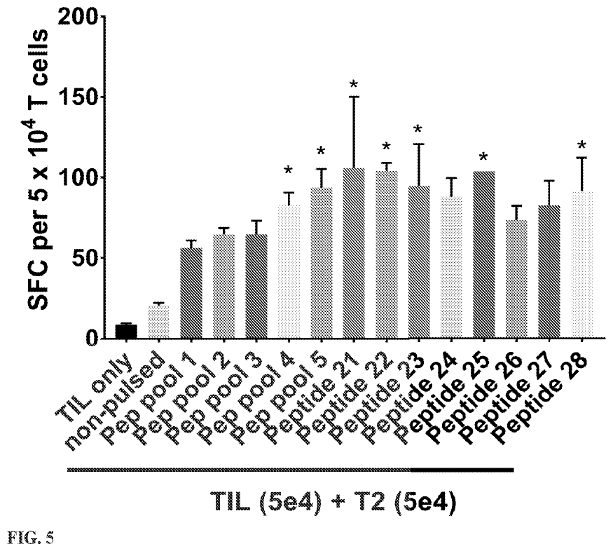

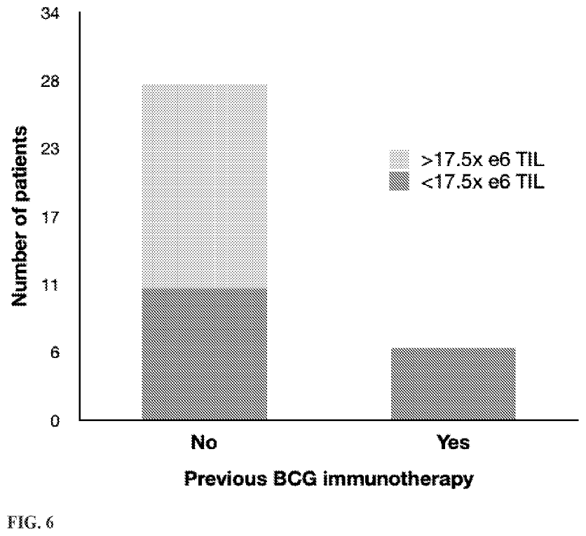

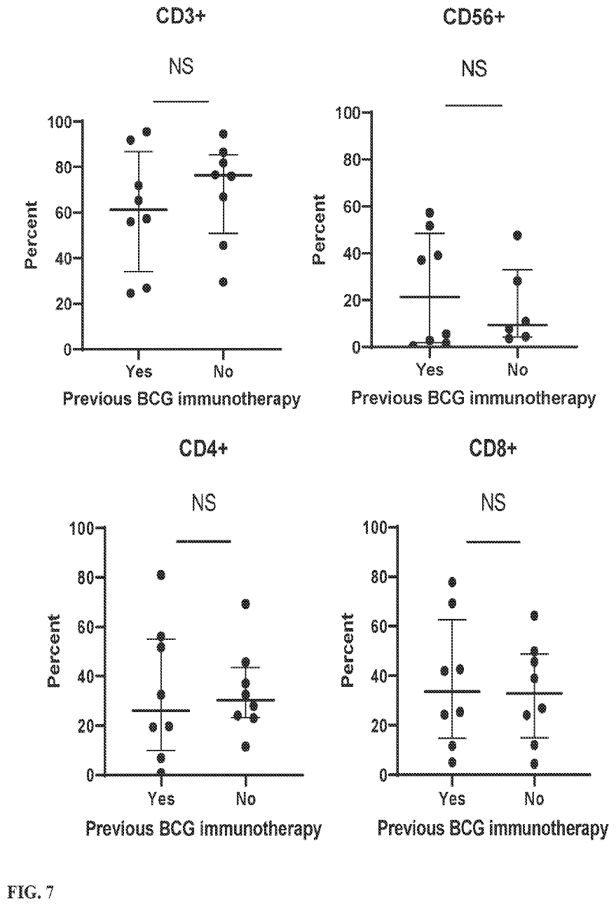

Most recently we have also demonstrated our ability to grow TIL from 6 previous BCG treated patients FIG. 6. While the total volume of TIL is lower it can still meet threshold for REP. We also demonstrated that the composition of immune cells within the tumor microenvironment was similar between the previous BCG exposure and no previous BCG exposure FIG. 7. This demonstrates the feasibility of harvesting TIL from patients of the target population.

While direct intravesical delivery of TIL can preclude the requirement for lymphodepleting chemotherapy, suppressive populations within the tumor microenvironment can contribute to suppression of transferred TIL. In experiments, we collected urine from 4 bladder cancer patients undergoing radical cystectomy. Immune cell subsets in the urine were measured by flow cytometry. As shown in FIG. 8, urine from bladder cancer patients contained a high number of myeloid derived suppressor cells (MDSC) and some T cells. MDSC have been shown to suppress T cell responses. MDSC were purified from the urine and co-cultured at various ratios with healthy donor T cells stimulated with anti-CD3 antibody. As shown in FIG. 8B, addition of MDSC highly suppressed the ability of T cells to secrete IFN-gamma. Although it is not known whether the MDSC in the urine originated from the tumor, this data suggests that there is a highly suppressive environment in the bladder of patients.

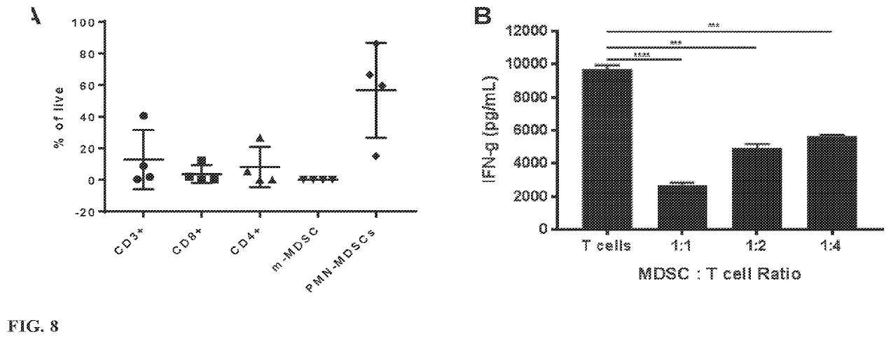

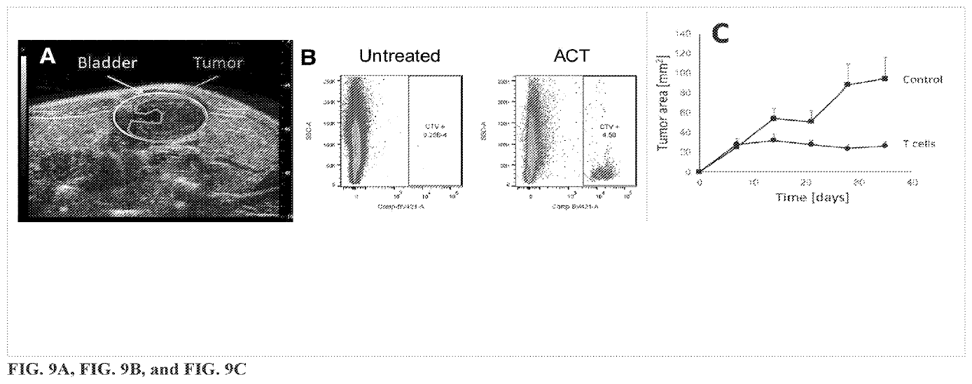

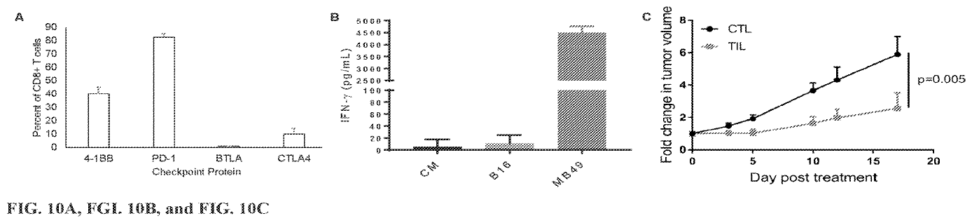

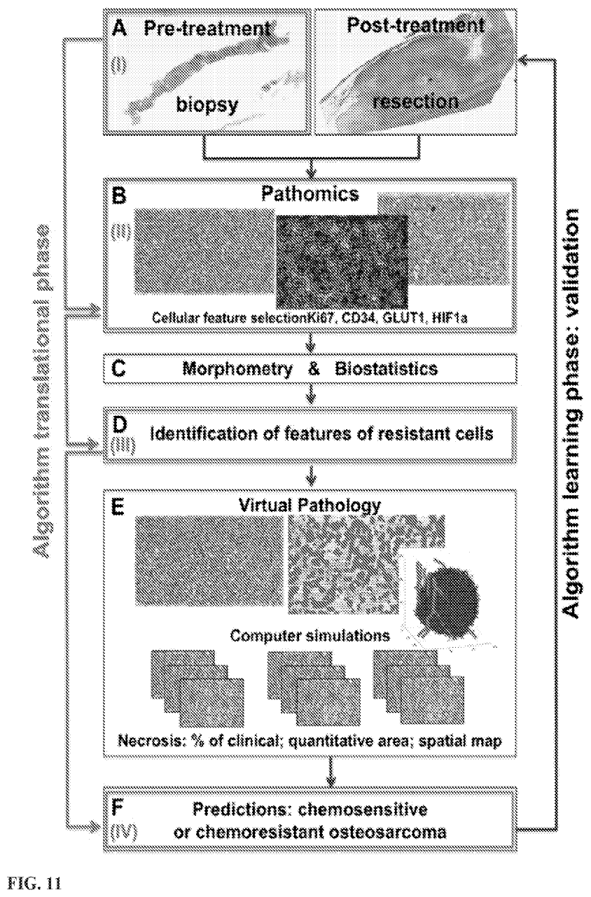

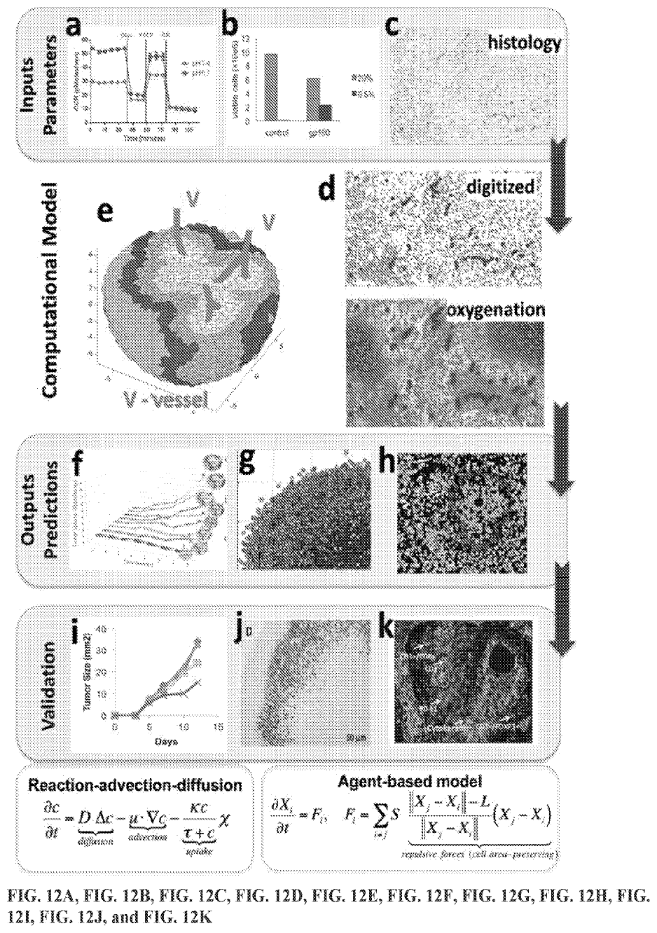

We next demonstrated the feasibility of intravesical (InV) ACT in a murine model. C57BL/6 mice were infused with 1×10⁵ MB49-OVA cells via bladder catheterization. One week later, tumors were detected by ultrasound (FIG. 9A). Under anesthesia, mice were infused inV via bladder catheter with 5×10⁶ OT-I T cells labeled with Cell-Trace Violet (CTV) dye, in the absence of pre-conditioning chemotherapy to induce lymphopenia. After 3 hours, mice were euthanized and bladder tumors were collected and digested to a single cell suspension. FIG. 8B shows that CTV+ T cells can be detected within bladder tumors. In additional mice, growth of ortho-topic bladder tumors was measured weekly by ultrasound. Figure C shows that intravesical infusion of T cells prevented tumor progression (p<0.05). While an OVA-based tumor model can be used to optimize treatment strategies, OVA is a highly immunogenic foreign antigen and does not represent the antigens found in patient tumors. We next developed a TIL ACT strategy in the relevant MB49 (non-OVA expressing) bladder tumor model. To demonstrate that tumors are infiltrated by CD8⁺ T cells, mice received MB49 bladder tumor cells subcutaneously (SC) on the right flank. After reaching 100-200 mm², tumors were resected and digested to single cells. Flow cytometry was performed to measure the immune cell subsets (phenotypes) infiltrating the tumor. We found 20% of infiltrating immune cells the tumor were CD8⁺ T cells. The CD8⁺ T cells isolated from tumors demonstrated expression of various checkpoint molecules, including 4-1BB and PD-1 (FIG. 10A). These cells were purified from tumors and expanded for one week in culture in high-dose IL-2. Expanded T cells were co-cultured in complete media (CM) alone, with irrelevant B16 melanoma cells, or with MB49 cells for 24 hours. Supernatants were collected and IFN-gamma was measured by ELISA. As shown in FIG. 10B, expanded MB49 TIL specifically recognized MB49 cells. Expanded TIL were infused into the bladders of mice bearing orthotopic MB49 tumors. Infusion of TIL led to a decrease in tumor volume (FIG. 10C), demonstrating the feasibility and efficacy of intravesical delivery of TIL. It is important to note that the effect of intravesical ACT with TIL was performed in the absence of lymphodepleting chemotherapy, supporting that pre-conditioning chemotherapy is not needed for effective TIL activity during local administration.

### a) Research Strategy

(1) To Perform a Phase I/II Clinical Trial of Intravesical Delivery of TIL in Patients with Bladder Cancer.

This novel trial can evaluate the safety and feasibility as primary endpoints. Recurrence and progression free survival can be secondary endpoints. Here we assess toxicity and efficacy of delivery intravesical TIL therapy in BCG unresponsive patients. We predict that patients intravesical TIL delivery is feasible with low toxicity profile. Secondary endpoints include recurrence free, progression survival and overall survival. This data can allow us to determine whether a trial to assess efficacy is safe to explore.

### (2) Study Design: Phase I/II Trial:

Twelve patients who meet the inclusion and exclusion criteria with NMIBC BCG unresponsive tumors can be screened and enrolled in the trial. Patients can be identified initially in the outpatient clinic. Based on the need for routine resection and/or biopsy patients can be identified by PI or co-I within the Genitourinary Oncology Program at Moffitt Cancer Center. Patients can complete baseline cross sectional imaging, urinalysis, complete blood count and metabolic panel as well as Bladder Cancer Index and AUA symptom score surveys. Patients can then undergo standard of care resection and/or biopsy. Tumors not required for clinical diagnosis, staging and treatment, can be used for TIL growth and expansion based on Moffitt Cell Therapies SOP. Peripheral blood and urine can also be collected for corollary studies at the time of surgery and prior to each TIL infusion. Based on experience we anticipate that TIL growth can be successful in nine of twelve patients. Tumor specimens that have successful TIL growth can go on to rapid expansion (REP). Autologous TIL can then be divided into 4 doses and infused intravesically once a week for a total of four weeks. Once a week dosing is in line current standard of care practices for intravesical immunotherapy and chemotherapy. The dose to be administered is up to 3.2 cells per 40 mls×4 doses. This equals 1×e9 cells in total which we can obtain from a REP flask with a total volume of 40 mL. Similar to current practice with intravesical immunotherapy and chemotherapy dwell time can be up to two hours. Intravesical therapy can be administered via gravity instillation. Patients can be monitored during treatment with q 15 minute vital signs and CTCAE v5.0 assessments for any serious adverse event SAE. Patients can also be monitored four hours post delivery to assess for tolerability and SAE. Supportive care during and after infusion can be provided with standard analgesic, anti-pyretic, anti-cholinergic medications. Patients unable to hold infusion therapy in the bladder or void spontaneously during treatment can be recorded. These patients can be included in the intention to treat analysis. Patients can be queried for SAEs and AUA symptom score each post treatment day by clinical trial coordinator. At 12 weeks after first infusion patients can undergo clinic visit, urinalysis, cystoscopy and cross sectional imaging. Patients can be assessed with CTCAE, AUA symptom score, and Bladder Cancer Index.

### (3) Primary Endpoint

The primary endopoint of the trial can be efficacy of TIL growth and toxicity of intravesical therapy. Feasibility of TIL growth can be assessed by the ability to grow enough TIL to have adequate volume to delivery 4 doses of intravesical therapy. Tumors that fail to yield growth either by paucity of cellularity or by contamination can be considered growth and expansion failures. Based on our experience we expect growth and expansion of approximately 70-75%. Patients whose tumor does not generate enough TIL for therapy can be referred to their treating physician for standard therapy. Patients who cannot tolerate infusion with spontaneous voiding can be refered to their treating physician for standard therapy.

Toxicity of therapy can be assessed at the time of instillation and during clinic visits with history and physical, cystoscopy and patient reported outcomes using the CTCAE v5 at routine intervals. Bayesian toxicity monitoring plan (1e) can be used to continuously monitor toxicity events. The trial can be stopped if an excessive toxicity rate is observed. Treatment can be followed by routine cystoscopic evaluation to assess treatment response in line with secondary endpoints.

### (4) Secondary Endpoint: Recurrence Free Survival, Progression Free Survival, and Overall Survival:

At 3 months after initiation therapy patients can undergo cystoscopy and cross sectional imaging to assess for recurrence. If tumors are present patients can undergo tumor resection in the operating room as per standard of care. Patients with recurrent tumors of the same stage at trial enrollment or lower (e.g. Ta to Ta or Tis) can be considered recurrence. Patients with tumors of higher stage than at tumor enrollment can be considered progression. Mortality and cause of mortality can be recorded at the time event. Recist 2.0 criteria can be used to measure tumor recurrence on cross sectional imaging.

### (5) Antigens Recognized by TIL in Bladder Tumors.

While the presence of T cells within bladder tumors is associated with improved outcomes in patients, little is known about the specific antigens recognized by these T cells. Herein we show that expanded TIL from bladder tumors respond to patient-specific neoantigens.

### (6) Study Design:

Patients for which TIL, urine, peripheral blood, and tumor samples are stored in the laboratory (6 BCG refractory and 12 BCG naïve) as well as up to 12 trial patients can be included in these studies to define specific antigens recognized by infiltrating T cells. Characterization and function of bulk TIL samples can be performed. Whole exome sequencing can be performed on peripheral blood mononuclear cells and tumor tissue to characterize patient-specific mutations. RNA sequencing can be performed to predict which neoantigens are expressed as potential immune targets.

### (7) Analysis of TIL Phenotype and Function:

A host of T cell phenotypic, differentiation, costimulatory, and co-inhibitory markers, including CD3, CD4, CD8, CD45RA/RO, CCR7, CXCR3, and other chemokine receptors, CD27, CD28, CD56, CD57, PD-1, BTLA, TIM-3, LAG-3, CTLA-4, CD25, CD69, CD103, 41BB, OX40, KIRs, KLRG1, cell cycle regulators, apoptosis regulators, STAT factors, T-bet, eomesodermin, different forms of Granzymes, Perforin can be measured on post-REP TIL products. We have developed different 8-12 color panels for analysis on a FACSCelesta cytometer (BD Biosciences). These data can be used to determine whether specific T cell phenotypes are predicted by mutation status and/or can predict the expansion of TIL from tumors. To facilitate data analysis, we can use CytoBank™ software tools70 as well as a new form of spatial flow cytometry data analysis called “Spanning-tree Progression Analysis of Density-normalized Events” (SPADE) specifically developed for larger multi-color staining panels. To measure tumor-reactivity, TIL can be co-cultured at a 1:1 ratio with autologous tumor cells and autologous tumor cells that are stained with anti-MHC class I antibody to block MHC class I presentation of antigens. Controls can include TIL alone (negative control) and TIL cultured with anti-CD3 antibody to induce maximum activation (positive control). After 24 hours, supernatants can be collected. IFN-gamma, TNF-alpha, IL-2, and Granzyme B can be measured by ELLA.

### (8) Whole Exome Sequencing:

DNA isolated from tumors and PBMC can be subjected to whole-exome sequencing. Whole-exome sequencing can be performed by the Molecular Biology Core at Moffitt Cancer Center in order to identify somatic mutations in the coding regions of the human genome. Two hundred nanograms of DNA can be used as input into the Agilent SureSelect XT Clinical Research Exome kit, which includes the exon targets of Agilent's v5 whole-exome kit, with increased coverage at 5000 disease-associated targets. Briefly, for each tumor DNA sample, a genomic DNA library can be constructed according to the manufacturer's protocol and the size and quality of the library can be evaluated using the Agilent BioAnalyzer. Equimolar amounts of library DNA can be used for a whole-exome enrichment using the Agilent capture baits, and after quantitative PCR library quantitation and QC analysis on the BioAnalyzer, approximately 100 million 75-base paired-end sequences can be generated using v2 chemistry on an Illumina NextSeq 500 sequencer. Mutational analysis can be performed to determine the number of neoantigens within tumors in collaboration with the Bioinformatics Core at Moffitt Cancer Center. Briefly, sequence reads can be aligned to the reference human genome (hs37d5) with the Burrows-Wheeler Aligner (BWA), and duplicate identification, insertion/deletion realignment, quality score recalibration, and variant identification were performed with PICARD and the Genome Analysis ToolKit (GATK). All genotypes (even reference) can be determined across all samples at variant positions using GATK. We can also sequence 60 patient-matched PBMC samples using the same procedures in order to remove artifacts and other false positives common to both tumor and normal samples. Various quality control measures can be applied to determine depth of coverage in each sample across the targeted genes. Sequence variants can be annotated using ANNOVAR, and summarized using spreadsheets and a genomic data visualization tool, VarSifter. Additional contextual information can be incorporated, including allele frequency in other studies such as 1000 Genomes and the NHLBI Exome Sequence Project, in silico function impact predictions, and observed impacts from databases like ClinVar and the Collection Of Somatic Mutations In Cancer (COSMIC). We can determine whether TIL expansion is associated with neoantigen numbers within individual patients and whether shared neoantigens are present among multiple patients.

### (9) Identification of Neoantigens Recognized by TIL:

Mutated epitopes expressed by autologous tumors can be defined. Using the whole exome sequencing analysis a peptide-binding algorithm can be used to predict potential epitopes restricted to the individual patients' HLA class I alleles. RNA-seq analysis from the patient tumor sample can also be performed by the Moffitt Molecular Biology Core Facility to limit the number of candidate peptides to those derived from expressed gene products. RNA can be extracted and can be processed for RNA-seq using the NuGen Ovation Human FFPE RNA-Seq Multiplex System to assess differential gene expression. Briefly, 100 ng of RNA can be used to generate cDNA and a strand-specific library following the manufacturer's protocol. Quality control steps including BioAnalyzer RNA chip runs and quantitative RT-PCR for library quantification can be performed. The library can be sequenced the Illumina NextSeq 500 sequencer with a 75-base paired-end run in order to generate 40-50 million read pairs. Sequence reads can be aligned to the human reference genome (hs37d5) using Tophat2. Aligned sequences can be assigned to exons using the HTseq package against RefSeq gene models to generate initial counts by region. Normalization, expression modeling, and difference testing were performed using DESeq2. RNAseq quality control includes in house scripts and RSeqQC to examine read count metrics, alignment fraction, chromosomal alignment counts, expression distribution measures, and principal components analysis and hierarchical clustering to ensure sample data represents experiment design grouping. Up to 200 mutated peptides that are predicted to bind with high affinity to the patients' HLA type can be synthesized. Peptides can be pulsed onto autologous PBMC or B cells for co-culture with expanded TIL. Tumor-reactive T cells can be isolated after a 12 hour co-culture with peptide-pulsed antigen presenting cells by sorting on CD3+ T cells that upregulate OX40 or 41BB. Sorted cells (positive and negative fractions) can be expanded and recognition of individual peptides can be evaluated using the ELLA platform to measure IFN-gamma, TNF-alpha, and Granzyme B. These studies can allow us to determine whether TIL products contain neoantigen-reactive T cells, the percentage of neoantigen T cells within TIL products, and determine whether enrichment of neoantigen-specific TIL can be beneficial for future clinical trials.

### (10) Analysis of T Cell Repertoire and Persistence of T Cells:

As intravesical therapy with TIL is completely novel, it is unclear whether infused T cells can persist at the tumor site or in peripheral blood. To evaluate whether TIL is present in urine or blood after intravesical infusion, TIL infusion products can undergo TCR-beta sequencing to define the T cell reperatoir in the TIL product. Blood and urine can be collected from patients at time of surgical resection and prior to each TIL infusion. T cells in urine and peripheral blood at each timepoint, as well as a sample of the TIL infusion product can be shipped to Adaptive Biotechnologies for T cell repertoire analysis using the ImmunoSEQ platform. The overlap of the TCR repertoire in the TIL infusion product can be compared to T cells in the peripheral blood and urine at each timepoint to determine whether unique clones in the TIL product are detectable within the periphery or urine. Positive results can allow us to determine the persistence of intravesically infused TIL and potentially correlate persistence with efficacy.

(11) an in Silico Model that Predicts Patients' Benefit from TIL Therapy.

While our study showed that about 70% of collected primary bladder tumors demonstrated TIL expansion, this process lasts up to four weeks, thus being able to predict early, at the time of diagnosis, whether or not the patient can benefit from TIL therapy can help in taking decision about an alternative treatment.

### (12) in Silico Analysis of Tumor Morphology:

The collected specimens from a cohort of 53 patients demonstrated that there was no significant difference between tumor subtypes in terms of viable and reactive TIL growth. Our goal here is to examine this data in terms of tissue metabolic environment and to correlate it with TIL expansion. These samples together with additional tumor samples stored in the laboratory (6 BCG refractory and 12 BCG naïve) can be used for morphological analysis. Histologic samples of FFPE tissue sections (4 μm) stained with H&E and IHC (for vasculature and immune cells) can be digitally scanned using the Aperio XT high-throughput slide scanner and segmented with Definiens TissueStudio software (Moffitt Analytic Microscopy Core). The machine learning-based in house algorithms of Landscape Pathology approach can be used to automatically identify tumor regions of interest and to quantify morphological and immunohistochemical features (50 for each segmented cell).

### (13) In Silico Predictions of In Vivo Tumor Response to Treatments.

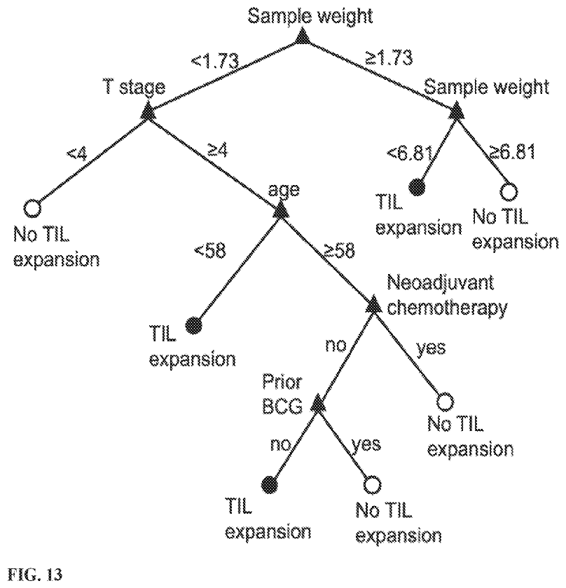

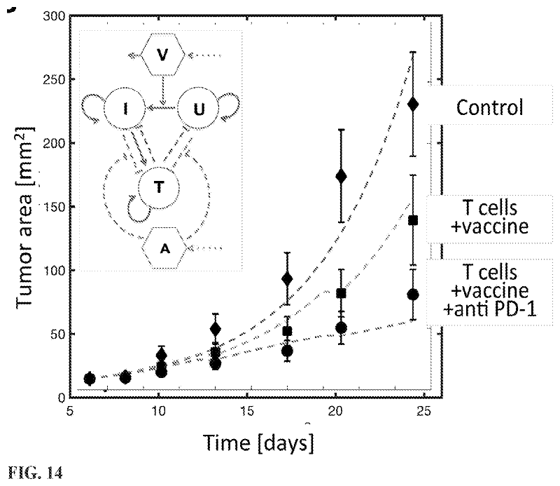

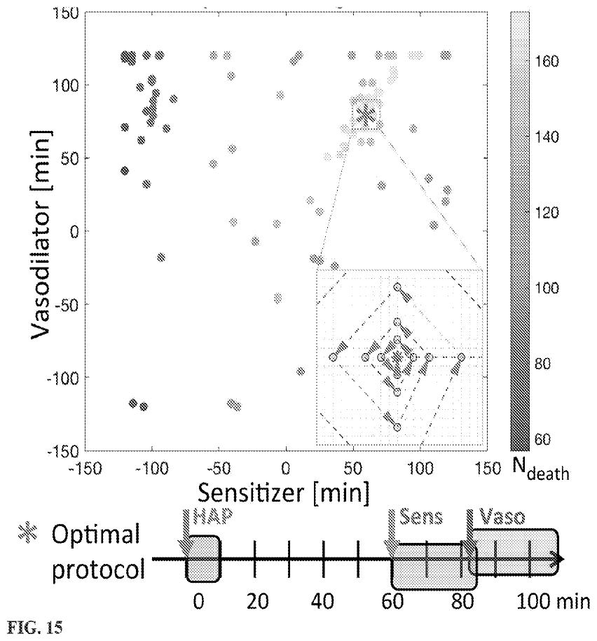

In vivo microenvironments are complex and difficult to recreate in laboratory in a controlled way, but manageable to in silico modeling. We developed a micro-pharmacology model to simulate in vivo tumors using both in vitro measurements and ex vivo tumor histology (FIG. 13a-c). The digitized tissue image contains locations of tumor vasculature, tumor, immune, and other stromal cells. It is used as a basis to predict kinetics and distributions of various metabolites, such as acid, oxygen or lactate and to reconstruct the 3D tumor (FIG. 13d,e). The model includes diffusive and advective transport of metabolites, migratory TILs (modeled as single agents) and other cell populations identified experimentally, as described above. Model outputs include growth response curves, cell-level data in IHC and fluorescent images that can be directly compared to experimental measurements (FIG. 13f-k). This approach was previously used to predict penetration of targeted imaging agents and short-term fluctuations in tissue oxygenation. Here, we can combine this model with the model of 3D tumor spheroid growth and infiltration by the immune T cells.

(14) in Silico Experiments for Correlation with TIL Growth:

For the already collected data patients from and additional 18 stored in the lab), we can simulate the distributions of oxygen, pH, and lactate within the tumor based on tissue vascular structure and cellularity assessed from histology images. Next, we can perform spatial morphometry and landscape ecology analyses (including metrics of diversity, dominance, or edge density) to determine patterns of immune cell infiltration in relation to regions of normoxia vs. hypoxia, and neutral vs. acidic microenvironment in which the TIL reside. To compare classification results for different histology images we can (i) test feature distributions with the Kolmogorov-Smirnov criterium; (ii) compare localization of cellular phenotypes using the density of states function; and (iii) correlate the cumulative statistics of tissue samples with recorded TIL expansion. We can achieve at least 90% power to detect a true difference with effect size of 0.7 for each quantitative image feature with false discovery rate (FDR) of 0.05 using a two-sided t-test. This estimate assumes that at least one of the 50 features can be differentiated between groups (˜25 features per group).

(15) Develop an in Silico Classifier for Predicting Weather Patient can Benefit from TIL Therapy:

Following the schematic of Virtual Clinical Trial predictor discussed in data, we can design the Learning phase of the algorithm using the already collected data from 53 patients, and can augment this data with analyses of tumor morphology and in silico simulations of tumor metabolic landscape, as described above.

Our analysis of collected specimens included the following clinicopathological variables: age of a patient at the time of surgery, sample weight, tumor grade, clinic stage, prior treatments, and histology classification, as well as TIL expansion status identified with in vitro cultures. The multilevel binary classification tree model has been constructed (FIG. 13) and trained on the 38 patient's data (out of 53) for which all clinicopathological inputs were available, yielding the concordance index (c-index) of 0.95. However, this model shows that the sample weight is the most critical covariate, followed by the clinical stage, and age at the surgery. We envision that by incorporating information about spatial distributions of TIL within the tumor tissue and tissue metabolic landscape for all 53 patients, we can design classifier of a higher prediction power with more pronounced contribution from other covariates. We can test this classifier using cross-validation methods, and compare classifier performance using the Receiver Operating Characteristic (ROC) curves.

### (16) Testing in Silico Model Predictions:

The second stage of the Virtual Clinical Trial predictor development is its validation with an independent set of data. We can utilize data from tumor collected. Whenever a new data can be available, the tumor grade, clinical stage and histology classification can be determined. A section of tumor can be used for staining, digitally scanning, and advance image analysis to identify TIL patterns, as well as to computationally simulate tumor metabolic landscape. This information can be used to predict the ability of TIL expansion based on their clinico-pathological and immunohistochemical data. In parallel, tumor specimens can be minced into fragments and cultured to determine TIL expansion in vitro. Cultures that expanded past 2 wells for any fragment can be considered positive for TIL growth. Subsequently, we can compare the predicted with actual TIL expansion to assess predictor sensitivity and specificity. Every half a year, we can recalibrate the in silico model with new data to improve its performance. This allows us to predict the likelihood of TIL expansion for a given tumor, and can provide a verified predictor tool that identifies a subset of patients that would benefit from ACT with TIL.

### 2. Example 2: Develop and Validate an in Silico Model to Enhance T Cell Infiltration into the Bladder Tumor

Rationale: The success of the first phase of ACT-TIL therapy in clinic is related to the number of tumor-specific T cells that are contained in the resected tumor. The more tumor-reactive T cells able to infiltrate the tumor before resection, the higher chance of subsequent T cell expansion ex vivo. Our recent studies with bladder tumor specimens obtained from chemotherapy-naïve cases showed that about 70% (14/20) of specimens led to T cell expansion. Here we develop and validate a mathematical model capable of predicting intervention protocols to increase T cell infiltration into the tumor or T cell expansion at the tumor site. We can first characterize the cellular composition of orthotopic tumors to determine cell populations to be included in the in silico model. The model can be subsequently used to predict optimal protocols for combined therapies to increase T cell infiltration that can be validated in the mouse orthotopic model of a bladder cancer. Finally, we can provide a testing software.

### a) in Silico Model of Multi-Treatment Combinations.

Temporal evolution of tumor size a result of interactions between tumor cells and T cells under various therapeutic treatments can be described by a system of coupled ODEs. FIG. 14 shows a case when TIL, PD-1 checkpoint inhibitor and cancer vaccine are combined. Five ODEs define behavior of untrasfected tumor cells (U, Eq.1.1), tumor cells transfected with the vaccine (I, Eq.1.2), T cells (T, Eq.1.3), vaccine (V, Eq.1.4) and anti-PD1 (A, Eq.1.5), with a total of 8 model parameters. The corresponding interaction flowchart is shown in the inset of FIG. 14. These equations were fitted hierarchically to match three sets of experimental measurements of tumor size (FIG. 14, black dots+/−SEM, standard error of the mean), step 1: tumor and T cells without treatment (FIG. 14 control, blue line), step 2: with a cancer vaccine (FIG. 14, green line), and step 3: with cancer vaccine and PD-1 checkpoint inhibitor combined (FIG. 14, red line).

$$\frac{dU}{dt} = {\underbrace{rU}}_{proliferation} - {\underbrace{{k}_{V} \frac{UV}{U + I + T}}}_{transfection} - {\underbrace{{c}_{T} \frac{UT}{U + I + T}}}_{death by T Cells} \qquad ( 1.1 )$$

$$\frac{dI}{dt} = {\underbrace{rI}}_{proliferation} + {\underbrace{{k}_{V} \frac{UV}{U + I + T}}}_{transfection} - {\underbrace{{c}_{T} \frac{UT}{U + I + T}}}_{death by T Cells} \qquad ( 1.2 )$$

$$\frac{dT}{dt} = {\underbrace{{r}_{T} T}}_{prolif} + {\underbrace{{r}_{T I} I}}_{recruit} - {\underbrace{( {c}_{UI} - {c}_{A} ( U + I ) ) \frac{( U + I ) T}{U + I + T}}}_{regulation by tumor \& APD 1} \qquad ( 1.3 )$$

$$\frac{dV}{dt} = {\underbrace{{u}_{v} ( t )}}_{injection} - {\underbrace{{k}_{V} \frac{UV}{U + I + T}}}_{transfection} - {\underbrace{{d}_{v} V}}_{clearance} \qquad ( 1.4 )$$

$$\frac{dA}{dt} = {\underbrace{{u}_{A} ( t )}}_{injection} \qquad ( 1.5 )$$

The fitting was done using the Matlab® optimization routine fminsearch in order to minimize the L₂ norm □ (Eq.1.6) of the weighted differences between the averaged experimental data □^{exper} and the simulated results □^{model}, for the weighting, we normalized these differences by the SEM values at each time point to impose a better fitting to experimental data of non-uniform variability at different time points. To evaluate goodness of fit, we employed the statistical coefficient of determination R² (Eq.1.7), where

$$\overline{{Ξ}_{t}^{exper}}$$

is the average experimental data at time t.

$$ζ = \frac{1}{N} \sqrt[2]{{\sum }_{t} \frac{{( {Ξ}_{t}^{model} - {Ξ}_{t}^{exper} )}^{2}}{S E {M}_{1}^{2}}} \qquad ( 1.6 )$$

$${R}^{2} = 1 - \frac{{\sum }_{t} {( {Ξ}_{t}^{model} - {Ξ}_{t}^{exper} )}^{2}}{{\sum }_{t} {( {Ξ}_{t}^{exper} - \overline{{Ξ}_{t}^{exper}} )}^{2}} \qquad ( 1.7 )$$

### b) the MADS Optimization Technique.

Finding the most efficient schedule (the order of treatments, dosage, and time between each intervention) is crucial, however, a large number of potential schedule combinations makes it impossible for comprehensive experimental testing and requires fast numerical optimization methods. The Mesh Adaptive Direct Search (MADS,) is a rigorous method that utilizes a gradient-free approach which is preferable for computational models when the derivatives of modeled functions are often difficult to approximate. Moreover, MADS can be applied to both continuous and discrete models. These properties make MADS superior when compared to other direct search methods (i.e., coordinate search or generalized pattern search). We previously used MADS to design optimal treatment strategies for a combination of the hypoxia-activated pro-drug (HAP), a vasodilator (Vaso) and a metabolic sensitizer (Sens), (FIG. 3). Here, we can use MADS to determine administration schedules for vaccines and anti-PD1 that maximize T cell numbers within the tumor.

### c) Infiltration of TIL in MB49 Tumors.

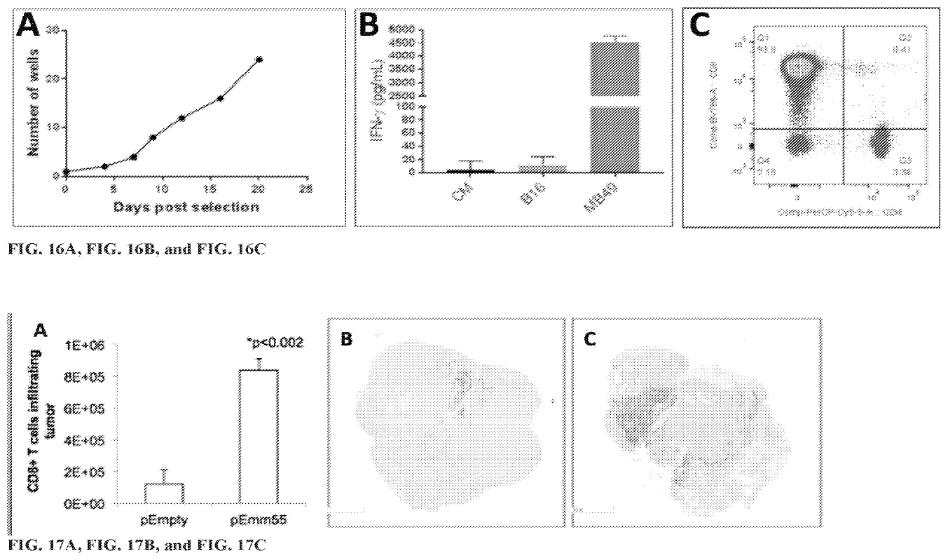

For our experimental model, we can use the murine MB49 bladder tumor cell line that has been shown to replicate well human urothelial carcinoma molecularly and phenotypically. We have demonstrated the ability to expand tumor-reactive TIL from orthotopic MB49 bladder tumors. Mice were inoculated with 1×10⁵ MB49-OVA cells intravesically (inV) after priming the bladder with poly-L-lysine as previously described. Tumors were dissociated and digested using a buffer containing Collagenase I, Collagenase IV, Hyalyronidase V, DNAse I and Hanks Buffered Saline Solution. T cells were isolated using CD90.2+EasySep positive selection. From the tumor digest, 1.65×10⁶ T cells were isolated and plated in 100 IU/ml of IL-2. After 4 weeks in culture TIL number increased to 39.2×10⁶ representing a 25-fold expansion over a three-week period (FIG. 16A). Expanded TIL produced a significant amount of IFN-gamma when co-cultured with MB49 cells but not in response to irrelevant B16 cells, indicating tumor-specificity of the expanded TIL (FIG. 16B). The phenotype of expanded bulk TIL: CD3+CD8+ (93.8%) and CD3+CD4+ (3.6%) T cells (FIG. 16C)

### d) T Cell Infiltration in Solid Tumors can be Enhanced by Stimulating Cancer Vaccines.

Emm55 is a serotyping protein normally expressed on the surface of the bacterium S. pyogenes. The use of emm55 as a priming antigen for the induction of tumor-specific immune responses has been shown in a clinical study in dogs in which the DNA plasmid containing the emm55 gene was transfected into canine lymphoma cells and used as a vaccine. We have shown that direct intralesional (IL) injection of a plasmid DNA vaccine (pAc/emm55) resulted in increased T cell infiltration in B16 tumors. Tumor bearing mice were treated with three IL injections of 20 mcg pAc/emm55 or empty plasmid DNA controls on days 7, 14, and 21 post tumor cell injection. Tumors were collected at day 7 after the final injection and T cells within the tumor were measured by flow cytometry (FIG. 17A), and by comparing immunohistochemistry (IHC) staining of control (FIG. 17B) or emm55-treated (FIG. 17C) tumors.

### e) Dendritic Cell Vaccine Delays Tumor Growth.

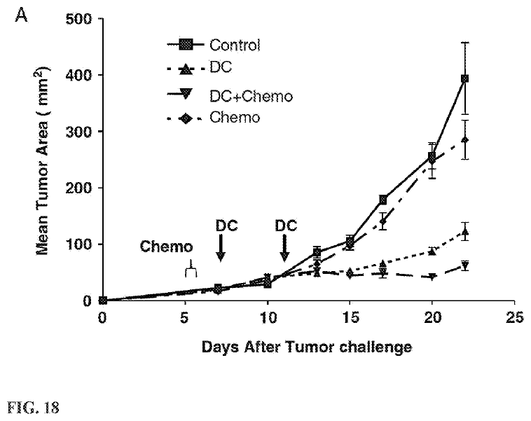

Dendritic cells (DC) are known as the most potent antigen-presenting cells, capable of initiating T cell immune responses. DC-based vaccines are comprised of ex vivo stimulated DC that are injected subcutaneously (s.c.) into the mouse. We previously tested the effects of DC vaccines on the growth of a murine model of melanoma (subcutaneous injection of M05 tumor cells. OVA-peptide pulsed DCs (1×10⁶) were injected at days 7 and 11 after tumor injection, and tumor size was monitored daily. As shown in FIG. 18, treatment with DC vaccine alone or in combination with chemotherapy (Cy and Flu injected at days 5 and 6, respectively), were able to delay tumor growth. We have also shown the efficacy of combination therapy with DC vaccination and Gemcitabine (Gem) to induce tumor regression in mice bearing pancreatic tumors.

### f) Characterize the Cellular Composition of the Orthotopic Bladder Tumors.

For these experiments, C57BL/6 mice can receive 1×10⁵ MB49 cells subcutaneously (s.c.) or into the bladder intravesically (InV) through catheters, after the bladder is treated with poly-L-lysine. Treatment can begin one week after injection when tumor volume is approximately 50 mm³. Mice can receive one of the following treatments alone or in combination: intralesional injections of emm55 plasmid one time per week for 3 weeks (control mice can receive empty plasmid), s.c. injection of DC pulsed with MB49 tumor lysate one time per week for 3 weeks (control mice can receive unpulsed DC), or intraperitoneal (IP) 20 mg/kg of anti-PD-1 (control mice can receive normal rat IgG). Tumor measurement can be recorded 2-3 times per week. In additional experiments, one week after the final treatment, tumors can be collected for flow cytometric analysis and IHC.

A portion of resected tumor can be digested into a single cell suspension for flow cytometry analysis of cell populations including tumor cells, myeloid cells (macrophage, MDSC, monocytes), and lymphocytes (CD4+ T cells, CD8+ T cells, regulatory T cells, B cells, NK cells). PD-L1 expression can be measured on tumors and myeloid subsets. We can further analyze T cells within tumors by flow cytometry using antibodies against a host of T cell phenotypic, differentiation, costimulatory, and co-inhibitory markers, including CD3, CD4, CD8, CD44, CD62L, CCR7, CXCR3 and other chemokine receptors, CD27, CD28, CD56, PD-1, BTLA, TIM-3, LAG-3, CTLA-4, CD25, CD69, 41BB, OX40, KIRs, KLRG1, cell cycle regulators, apoptosis regulators, STAT factors, T-bet, eomesodermin, different forms of Granzymes, Perforin. We have developed different 8-12 color panels for analysis on a FACSCelesta cytometer (BD Biosciences). These staining panels can be used to distinguish the frequencies of myeloid cells and lymphoid cells, and specific CD8+ and CD4+ T cell subsets based on co-expression of different markers. These data can be used to determine the set of cells (tumor, stromal, immune) to be included in the mathematical model. To facilitate data analysis, we can use CytoBank™ software as well as a new form of spatial flow cytometry data analysis called “Spanning-tree Progression Analysis of Density-normalized Events” (SPADE) specifically developed for larger multi-color staining panels.

### g) Quantitatively Extend the Macroscopic-Level Model for Testing Multi-Treatment Combinations.

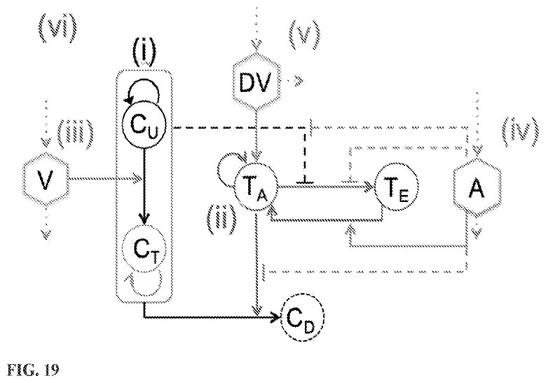

Based on the model in data, we can build the ODE models (i)-(vi) with schematics shown in FIG. 19 in a hierarchical way, so that the latter models can inherit components and parameters from the former models. In model (i), only the tumor growth (variable C=C_{U}+C_{T}) can be modeled, while in model (ii), the interactions between tumor cells (C) and T cells (active T_{A} and exhausted T_{E}) can be included. We can add: in model (iii) the stimulating emm55 vaccine (V) and transfected Cr tumor cells (Cᵤ-untransfected), in (iv) the PD-1 blockade inhibitor (A), in (v) the dendritic vaccine (DV), and in (vi) the combination of all therapeutic interventions. We can follow the calibration procedure as described in the data using the experimental results. We may extend the model by including other subpopulations of T cells based on the results of immune milieu analysis.

### h) Optimal Protocols for Combined Therapies to Increase T Cell Infiltration.

Shown herein is the determination of optimal treatment protocols with the following objectives: (a) maximizing number of T cells within the tumor; (b) minimizing tumor burden, (c) minimizing the number of therapeutic interventions. We can consider each objective separately, as well as their combinations. For the ODEs describing each model (i)-(vi) from FIG. 19, we can first perform parameter calibration to match the corresponding experimental data. The fitting can be done using the MATLAB optimization routine fminsearch to minimize the L₂ norm of the weighted differences between the averaged experimental data and the simulated results; for the weighting, we can normalize these differences by the SEM values at each time point. This can be followed by local sensitivity analysis of model parameters to identify tight credible intervals and possible parameter correlation. The goodness of fit evaluation can employ the statistical coefficient of determination R² and the fits for which R² is 0.9 or larger can be accepted. The models can be fitted hierarchically and the higher-level models can inherit parameters fitted in the lower-level models (similarly to data above).

Next, we can design optimal treatment protocols for each model. The final full model can include three types of therapeutic interventions: stimulating vaccine emm55, dendritic vaccine, and PD-1 checkpoint inhibitor. The treatment protocol variables include the order of treatments, timing of each injection and its dosage; each could potentially be varied over a large number of values. We can approach this by formulating a mixed-integer optimization problem with integer variables referring to the discrete schedule (time in minutes/hours/days) and the real-number variables referring to the continuous dosages (volume of injected treatments). These optimization problems can be solved using the MADS method (data) with search constrains that can ensure that the obtained optimal protocols are within biologically (and clinically) feasible values.

Since we postulate multiple objectives for the optimization process, we expect to observe competition between the objective functions. For example, the optimal protocol that minimizes tumor burden can most likely not be optimal with respect to minimizing number of interventions. Therefore, we can use the Pareto optimality principle to determine the trade-offs between each pair of objective functions through a set of multi-objective (MO) optimization simulations. MADS can be used to solve the MOs and generate solutions at the trade-off closest to the utopia point. The shape of the resulting Pareto fronts can be analyzed, and the regions where a substantial decrease in one objective function causes minimal compromise to the other objective functions can be recommended for experimental validation. As a result, we can obtain a set of protocols, each with different weights of treatment attributes (e.g., tumor burden vs. total number of treatment interventions). This can provide a pool of schedules for selection in collaboration with experimentalists and clinicians.

i) Validate T Cell Infiltration after Optimal Treatment.

We can follow the experimental procedure described herein, but with the administration schedule of emm55, DC and anti-PD1 predicted by our mathematical model. The resected tumors can be used to determine whether MB49-specific T cells have migrated into the tumor tissue. Tumors can be fixed and T cell markers can be measured by IHC staining. After tumor digestion, T cell markers, and markers for additional immune subsets (Tregs, MDSC, macrophage, NK cells) can be measured by flow cytometry. This can be compared to simulated cases in order to validate the extent of T cell infiltration.

### j) Provide a VirTuOSo Module for Schedule Testing.

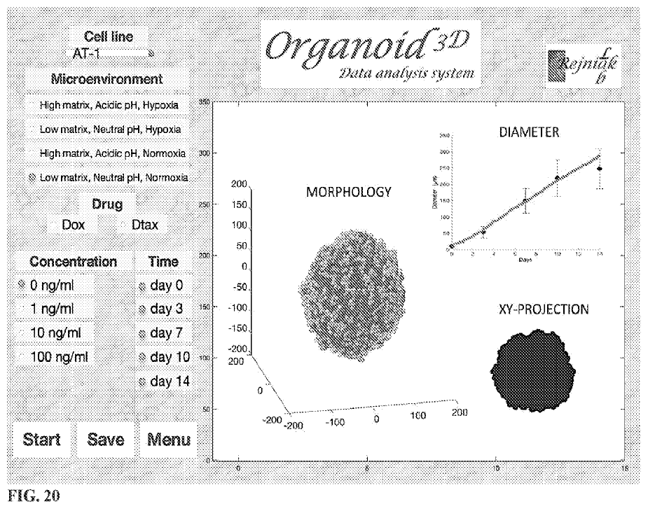

Enabling an easy use of the developed algorithms by both experimental and computational users allows for faster and more accurate data analysis, schedule testing, and generation of testable predictions. We can drawn on our past experience (FIG. 20) and utilize the MATLAB graphical user interfaces (GUI) system, that is versatile enough to work with various data formats: graphical files (jpg, tiff); text files, and Excel spread-sheets, and allows for both advanced programing and visualization of the obtained results.

The MATLAB-based GUI platform can include the following options: (1) input data of a time series of tumor sizes from in vivo experiments with and without treatment; (2) progressive data fitting to define parameters of the cell population model; (3) simulations of virtual treatment protocols; (4) determination of optimal protocols.

We can also take advantage of MATLAB capabilities to create a stand-alone executable software with Matlab Runtime application. This can enable the use of our analysis and simulation software on machines that do not have preinstalled MATLAB system. Furthermore, we can create an online version of our tool using MATLAB Web App Designer. This can enable easy access to our tool by other researchers.

### 3. Example 3: Predict in Silico and Validate in PDX Model the Methods to Enhance T Cell Functionality

For expansion of TIL, resected tumors are minced into fragments, placed into individual wells of a 24-well plate, and propagated in media containing 6000 IU/mL IL-2. Once the T cells in a single fragment well reach confluence, the cells are expanded into additional wells. TIL are expanded ex vivo for up to six-weeks prior to infusion into patients. This ex vivo expansion gives an opportunity to optimize T cell properties for the most effective T cell-tumor cell interactions after reinfusion. In this Aim, we can collect tumors from bladder cancer patients and evaluate their growth dynamics in different fixed microenvironmental conditions, quantify their secretome and histology, and use this data to calibrate the microscale mathematical model. We can simulate T cell functionality in dynamical microenvironments and validate model predictions in the PDX models. All computational algorithms can be combined into a module of the VirTuOSo software.

### a) Mathematical Model of Tumor Microenvironment and Micropharamcology.

The in vivo tumor microenvironments are complex and dynamically changing, and thus difficult to recreate in laboratory. However, they are manageable to in silico modeling. We previously developed a concept of micropharmacology modeling that allows for in silico investigation of the transport and actions of drug and metabolites within the explicitly defined tissue structure. This modeling framework was used to simulate tissue oxygenation, development of chronic and transient hypoxia regions and scheduling of hypoxia-activated pro-drugs; all modeled with continuous reaction-advection-diffusion equations. The micropharmacology framework was also used to model the distribution and uptake of targeted fluorescent imaging biomarkers; with the imaging agent molecules modeled as individual point-particles. In the current project, we can use the micropharmacology model to represent continuous concentrations of oxygen, acid and cytokines, and individual agents to represent T cell-based therapies. We can also incorporate in this model both in vitro single cell data and ex vivo tumor histology.

### b) Effect of Defined Metabolic Conditions on IFN-Gamma Secretion by T Cells.

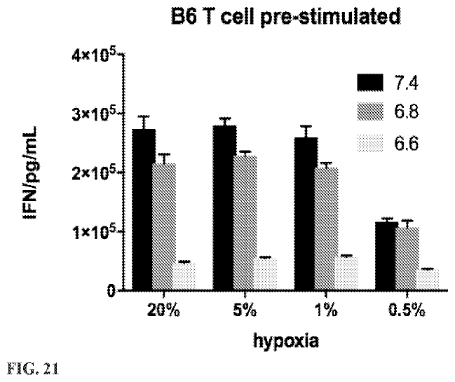

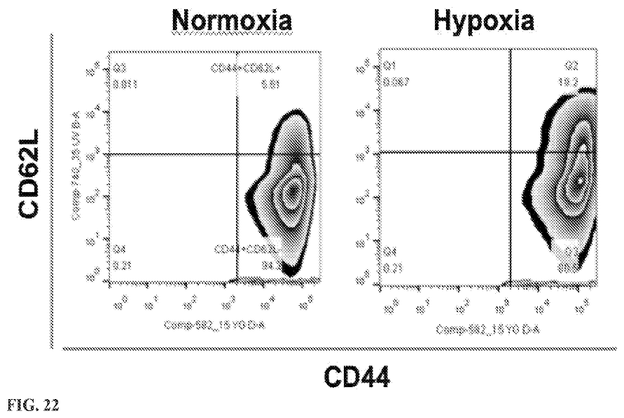

We have characterized changes in the production of IFN-gamma by T cells exposed to different oxygen and pH conditions in vitro. T cells isolated from the spleens of naïve C57BL/6 (B6) mice were cultured at 37° C. under a combination of the normoxic (20% O₂) or hypoxic (94% N₂, 5% CO₂, 1 or <1% O₂, Sanyo) conditions and under three levels of acidity (pH 7.4, 6.8 and 6.6) in the presence of anti-CD3/CD28 antibodies. Cell supernatants were collected at 48 hours and the secretion of IFN-gamma was measured by flow cytometry. As shown in FIG. 21, the production of IFN-gamma in B6 T cells was decreased in severe hypoxia and was uniformly low in acidic conditions. We further evaluated T cell phenotypes at 1% 02 at normal pH and measured an increased percentage of T cell with a memory phenotype (CD44+CD62L+, FIG. 22). These results indicate that acidosis and severe hypoxia promotes the down-regulation of T cell activity, but moderate hypoxia may enhance T cell function.

### c) Effect of Checkpoint Targeting on TIL Expansion.

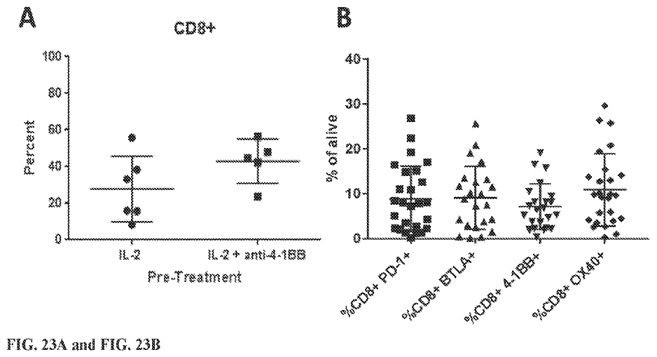

We have shown that addition of agonistic anti-41BB antibodies at the initiation of TIL outgrowth from tumor fragments had a beneficial effect on increasing CD8⁺ TIL and anti-tumor activity in melanoma. We also evaluated whether 4-1BB agonism could improve the expansion of T cells from primary bladder tumors. Addition of anti-4-1BB antibody to the culture media of tumor fragments led to an increase in the number of fragments with TIL expansion (FIG. 23A). Furthermore, we evaluated expression of other co-inhibitory and co-stimulatory receptors on CD8+ T cells isolated from human bladder tumors and measured expression of PD-1, BTLA, and OX-40 (FIG. 23B). We hypothesize that the targeting of these molecules can lead to enhanced TIL expansion, improve the output of CD8+ T cells, and increase anti-tumor specificity.

d) Evaluate TIL Expanded from Tumor Fragments.

Primary tumors can be collected from 10 bladder cancer patients under an IRB-approved protocol. We can evaluate the growth kinetics of TIL from fragments after culture in media containing 3000 IU/ml IL-2. Antibodies can be added to target PD1, BTLA, or OX40 alone or in combination with anti-41BB antibodies. Antibodies can be added at the initial set up of bladder tumor fragments and subsequently added each time the TIL cultures are fed with IL-2. Control fragments can receive IL-2 alone or IL-2 plus anti-41BB antibody alone. In addition, fragments can be cultured with CM+IL-2 at normal or 1% O₂ levels. The number of TIL can be counted on days 7, 14, 21, and 28 after culture initiation. The proliferation of TIL cultured with anti-PD1, BTLA, or OX40 antibodies alone or in combination with anti-41BB antibodies, or at hypoxic conditions, can be compared to TIL cultured with IL-2 alone. In addition to increased proliferation, the phenotype and functional activity of expanded TIL can be measured. Surface expression of CD3, CD8, CD4, CD62L, CD45RA, CD45RO, CCR7, 41BB, OX40, PD1, and BTLA can be measured by flow cytometry. Expression of the CD8⁺ factors granzyme B, perforin, and CD107a can also be measured.

### e) Quantify the Secretome and Gene Expression Profile of Interacting Cancer Cells and T Cells.

To evaluate the cytokines expressed by tumor-reactive T cells, TIL can be co-cultured at a 1:1 ratio with autologous tumor cells and autologous tumor cells that are stained with anti-MHC class I antibody. Controls can include TIL alone (negative control) and TIL cultured with anti-CD3 antibody to induce maximum activation. After 24 hours, supernatants can be collected. Cytokines can be measured by cytometric bead array and ELISA. Cytokines can include IFN-gamma, TNF-alpha, IL-2, IL-10, and IL-17. In collaboration with the Moffitt Genomics Core Facility, gene expression analysis can be performed on resected tumor and immune infiltrates using the Nanostring PanCancer Immune Profiling Panel that can detect 770 genes covering multiple immune cell subsets, signaling pathways, chemokines, and checkpoint proteins. This assay can allow for identification of additional immune subsets and secreted factors within tumors, and to determine which additional cell subpopulations and extracellular factors can be included in the mathematical model.

### f) Characterize the Histology of Bladder Tumors by Quantitative Imaging.

A portion of the resected tumor can be used for histological analysis. Samples of tissue sections (4 μm) stained with H&E and IHC (CD34 for vasculature, HIF-1 for hypoxia-inducible factor, CD3, CD4 or CD8 antibody for immune cells) can be digitally scanned using the Aperio XT slide scanner and segmented with Definiens TissueStudio software (available at Moffitt Analytic Microscopy Core). The machine learning-based in house algorithms of Landscape Pathology can be used to automatically identify tumor regions of interest and to quantify the numbers and spatial infiltration patterns of T cells. CD34 staining can be used to determine tumor tissue vascularization. Expression of PD-L1 on tumor cells and infiltrating immune cells can be measured.

### g) Develop in Silico Model of Bladder Tumor Microenvironment and Predict T Cell Functionality in the Heterogeneous and Dynamically Changing Conditions.

Following results in melanoma: ours that quantified functionality of T cells in different microenvironmental conditions (FIG. 21), and published by Gropper et al. that showed higher toxicity of T cells activated under hypoxia, rather than in normoxia, we can determine the most effective protocols for enhancing T cell functionality using microscopic in silico model based on our micropharmacology framework.

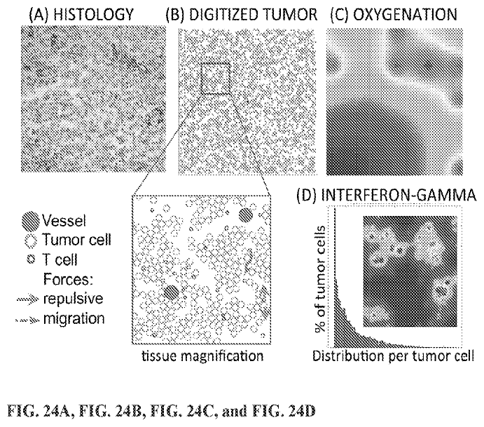

Our model (FIG. 24) is based on a digitized tissue histology (vasculature and tumor cell locations) and was used to predict kinetics and distribution of oxygen and interferon-gamma (IFN-gamma) within the tissue. This model includes diffusive transport of oxygen □ with supply S(t) from the vasculature Vₗ and uptake □x by tumor cells Xₖ and □_{Y} by immune cells Yₘ (Eq 2.1). The kinetics of IFN-gamma □ secreted by the immune cells Yₘ at a rate □ that depends on the level of oxygen in T cell vicinity (Eq 2.2). In hybrid models, the indicator function x links the discrete positions of cells Xₖ and Yₘ with continuous positions x of the metabolites that fall within a neighborhood with a radius R (Eq 2.3). The interstitial fluid flow u is modeled using the fluid-structure-interactions method of regularized Stokeslets (Eqs.2.4-2.5) with fluid viscosity □ and fluid pressure p. Repulsive forces f_{k,j} with the spring stiffness F and resting length 2R (Eq. 2.6) are imposed between overlapping cells to restore the distance between their centroid to equal the cell diameter. Moreover, T cells are subject to drag forces of random orientation that □ represent their migration with speed □□ through the interstitial space (Eq.2.7). The movement of each cell is modeled using the overdamped oscillator equation with the damping coefficient (Eq 2.8).

$$\frac{\partial η ( x , t )}{\partial t} = {\underbrace{{D}_{η} Δ η ( x , t )}}_{diffusion} + {\underbrace{u ( x ) \cdot ∇ η ( x , t )}}_{advection} - {\underbrace{{a}_{X} η ( x , t ) {\sum }_{k} 𝒳 ( x , {X}_{k} ( t ) )}}_{tumor cell uptake} - {\underbrace{{a}_{Y} η ( x , t ) {\sum }_{m} 𝒳 ( x , {Y}_{m} ( t ) )}}_{T cell uptake} + {\underbrace{S ( t ) {\sum }_{l} 𝒳 ( x , {V}_{l} )}}_{vascular influx} \qquad ( 2.1 )$$

$$\frac{\partial γ ( x , t )}{\partial t} = {\underbrace{{D}_{γ} Δ γ ( x , t )}}_{diffusion} + {\underbrace{u ( x ) \cdot ∇ γ ( x , t )}}_{advection} - {\underbrace{{\sum }_{m} β ( γ ( x , t ) ) 𝒳 ( x , {Y}_{m} ( t ) )}}_{cellular secretion} \qquad ( 2.2 )$$

$$𝒳 ( x , Z ( t ) ) = \{ \begin{matrix}0 & if ‖ x - Z ( t ) ‖ < R \\ 1 & otherwise\end{matrix} \qquad ( 2.3 )$$

$$μ Δ u ( x , t ) = ∇ p ( x , t ) - f ( x , t ) \qquad ( 2.4 )$$

$$∇ \cdot u ( x , t ) = 0 \qquad ( 2.5 )$$

$${f}_{k , j} ( {X}_{k} , t ) = \{ \begin{matrix}F ( 2 R - ‖ {X}_{k} - {X}_{j} ‖ ) \frac{{X}_{k} - {X}_{j}}{‖ {X}_{k} - {X}_{j} ‖} & if ‖ {X}_{k} - {X}_{j} ‖ < 2 R \\ 0 & otherwise\end{matrix} \qquad ( 2.6 )$$

$${g}_{k} ( {Y}_{m} , t ) = ω [ \begin{matrix}cos ϕ \\ sin ϕ\end{matrix} ] \qquad ( 2.7 )$$

$${\sum }_{j} {f}_{k , j} ( {X}_{k} , t ) + {g}_{k} ( {X}_{k} , t ) = - υ \frac{{dX}_{k}}{dt} \qquad ( 2.8 )$$

Model outputs include cell-level information about: tumor composition (cell locations, cell types and states, FIG. 24B), tumor cell exposure to IFN-gamma and their cellular uptake (spatial and temporal distributions, FIG. 24D), and metabolic gradients within the tumor (FIG. 24C) that can be compared to tumor histology images.

Here, we can extend this model to include other metabolites (acid or lactate), cytokines, (granzymes, IL-2 or IL-10), and ratios between tumor cells, T cells and other stromal cells acquired from flow cytometry studies. We can perform in silico tests of various administration protocols-multiple injections, injections with different numbers of T cells, intravenous (i.v.) vs. intralesional (I.L) vs. intravesical (inV) TIL administrations. As the outcome of this model, we can use the ratio of dead to viable tumor cells. The most effective protocols can be tested experimentally in mouse models of bladder cancer. Mathematical results: can be compared to tumor histology stained for dead cells or to dead cell counts from flow cytometry analyses.

h) Evaluate ACT with TIL in a PDX Model

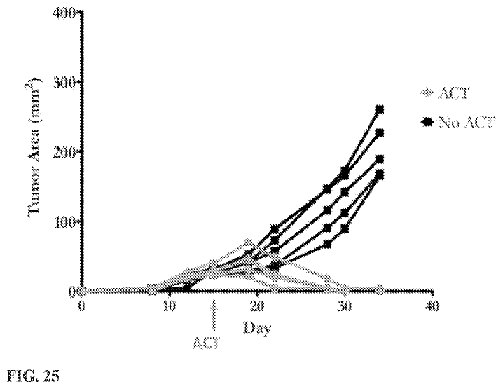

We have developed a protocol to establish bladder tumor patient derived xenografts (PDX) in immunodeficient NSG mice. We have also shown that treatment of PDX tumors with autologous TIL results in tumor regression (FIG. 25). In this experiment, NOG-IL2 mice were injected s.c. with 1×10⁶ patient-derived tumor cells. Once tumors reached 25-50 mm², mice were injected i.v. with 5×10⁶ patient-matched TIL (FIG. 25, ACT group). Control mice were untreated (FIG. 25, No ACT group). In this aim, fragments of patient tumors can be implanted into NSG mice. Once tumors are established, tumors can be digested and single cells can be injected s.c. or inV into NOG-IL-2 mice that constitutively express human IL-2. Mice can receive patient-matched TIL (i.v. or inV). Tumor growth can be measured and comparisons can be made between groups of mice that receive TIL grown under different conditions. We can also use this model to validate relevant in silico model predictions generated herein. At the end of the experiment, tumor histology can be quantitatively analyzed and used for comparison with the in silico model outcomes.

### 4. Example 4: Provide a VirTuOSo Module for Testing T Cell Functionality

The VirTuOSo module can allow for testing the extent (depth) of T cell infiltration and T cells functionality (secretion levels, interactions with tumor cells, killing potential) in diverse environmental conditions, and can include: (1) input data as a histology image; (2) quantitative feature extraction for tumor and stromal cells, and tissue vasculature; (3) input data from T cell secretome in various conditions as an Excel file; (4) simulations of tumor metabolic landscape; (5) simulations of immune cell infiltration and functionality; (6) predictions of cases with maximal gain.

### a) Optimize and Validate Combination Schedules of Adoptive T Cell Therapy in the Bladder Cancer

In clinic, TIL are usually administered intravenously. However, for patients with an intact bladder, treatments can be administered intravesically (inV) through a catheter. In contrast to systemic administration of TIL, this localized method allows for multiple TIL injections, and thus gives an opportunity to design novel mathematical model-based protocols. These can include intravesical ATC-TIL in combination with cancer vaccines, checkpoint inhibitors and gemcitabine (Gem) chemotherapy decreasing suppressive cell populations within the tumor microenvironment. The overall objective is to increase the effectiveness of reinfused TIL. In this aim, we can develop an in silico ACT-TIL model and the ACT-TIL in a syngeneic murine model, and use this integrated approach to validate in silico predictions. Finally, we can provide a software module for schedule testing.

### b) Murine Model of Intravesical ACT in Orthotopic Bladder Cancers.

We have demonstrated the feasibility of intravesical ACT using the MB49 cell line modified to express the model antigen, ovalbumin (MB49-OVA). The OVA MHC class I peptide SIINFEKL is recognized by OT-I T cells that are specific for the OVA_{SIINFEKL} peptide. To evaluate the feasibility of inV delivery of TIL, 1×10⁵ MB49-OVA cells were infused into the bladders of C57BL/6 mice via bladder catheterization as previously described. One week later, tumors were detected by ultrasound (FIG. 9A). Under anesthesia, mice were infused inV via bladder catheter with 5×10⁶ OT-I T cells labeled with Cell-Trace Violet (CTV) dye. After 3 hours, mice were euthanized and bladder tumors were collected and digested to a single cell suspension. FIG. 9B shows that CTV+T cells could be detected within bladder tumors. One week after inV infusion of MB49-OVA cells, tumors were detected by ultrasound and mice were randomized into 2 groups. Under anesthesia, mice were infused inV via bladder catheter with PBS or 5×10⁶ OT-I T cells. Growth of ortho-topic bladder tumors was measured weekly by ultrasound. FIG. 9C shows that intravesical infusion of T cells prevented tumor progression (p<0.05)

### c) Develop Macroscopic in Silico Model for Predicting Optimal ACT-TIL Protocols.

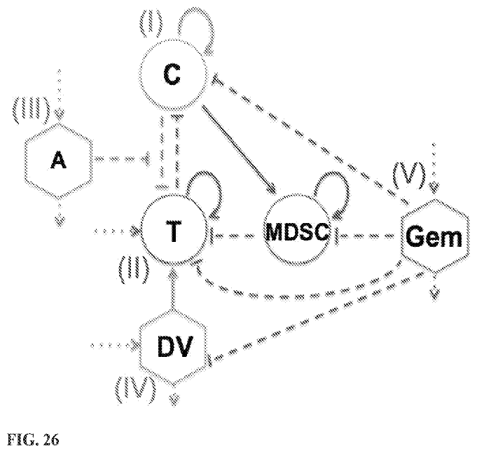

The mathematical models (FIG. 17, FIG. 19) can be adjusted to represent the biology of MB49 bladder cancer. The hierarchical structure of the ODE model (I-V) is shown FIG. 26, where model (I) includes tumor growth only (variable C); model (II) deals with tumor cells and TIL (T) interactions; in (III) the PD-1 blockade inhibitor (A) is added; in (IV) the dendritic vaccine (DV) is included; and in model (V) the chemotherapeutic agent Gem. Models (III)-(V) can require additional experimentation to analyze T cell-tumor cell interactions under the DV, A, and Gem treatment, respectively. For model (III), we can repeat experiments in FIG. 19 using MB49 cells, MB49-derived TIL cells and PD-1 blockade. For model (IV), we can repeat experiments and the treatment protocol as described in the data section (FIG. 9), but we can use bladder cancer MB49 cells, MB49 TIL and MB49 lysate-pulsed DC vaccines. For model (V), we can use Gem as our chemotherapeutic agent since it has been shown that Gem targets suppressive cell populations, such as myeloid-derived suppressor cells (MDSC). For each case, we can use the average experimental data for model calibration. We may also extend the mathematical model by including different sub-populations of T cells based on the immune milieu analysis.

For each calibrated hierarchical model, our goal is to determine optimal treatment protocols with the following objectives: (a) minimizing tumor burden, (b) minimizing the overall treatment dosage, (c) minimizing the number of therapeutic interventions, and (d) minimizing the number of injected TIL necessary to observe anti-tumor immunity. We can consider each objective separately, as well as their combinations. For the ODEs describing models (I)-(V), we can first perform parameter calibration to match the corresponding experimental data, as described in data. The fitting can be done using the MATLAB fminsearch routine, and parameters calibrated for a predecessor model can be fixed in the subsequent model extensions. Next, we can design optimal treatment protocols for the full final model with four types of therapies: TIL, dendritic vaccine, PD-1 checkpoint inhibitor, and MDSC cells-targeting chemotherapeutic agent Gem. The treatment protocol variables include the order of treatments, timing of each injection and its dosage. The MADS method can be used to solve this optimization problem, and we can use the Pareto optimality principle to determine the trade-offs between competing objective functions as described above.

d) Develop ACT with TIL in a Syngeneic Murine Model

While an OVA-based tumor model can be used to optimize treatment strategies, OVA is a highly immunogenic foreign antigen and does not represent the antigens found in patient tumors. One goal of this application is to develop a TIL ACT strategy in the relevant MB49 (non-OVA expressing) bladder tumor model. We have shown above (FIG. 16) that TIL can be isolated and expanded from MB49 tumors. For these experiments, orthotopic tumors can be established in C57BL/6 mice (CD45.2+) by injection inV of 1×10⁵ MB49 cells into the bladder. Donor TIL can be isolated from orthotopic MB49 tumors grown in congenic C57BL/6 (CD45.1+) mice. Recipient mice can be treated with 5-10×10⁶ TIL InV by catheterization one time or at weekly intervals for up to 6 weeks. Growth of orthotopic tumors can be monitored by ultrasound. In additional experiments, tumors can be collected at various time points after TIL delivery (1, 3, 7 and 14 days and at endpoint) for IHC, flow cytometric and functional assays as described herein.

The model can be further optimized by including strategies to potentially decrease suppression at the tumor site or enhance TIL reactivity. MB49 tumors can be established in C57BL/6 mice. One week later mice can be infused inV with PBS or 5×10⁶ MB49 TIL cells. We can evaluate 3 approaches: 1. To decrease T cell suppression, starting at day 8, mice can receive intraperitoneal injection of 15 mg/kg of either isotype NrIgG control antibodies or anti-PD-1 blocking antibodies twice per week; 2. To enhance reactivity of transferring TIL, mice can be treated with MB49 lysate-pulsed DC injected s.c. on days 8, 10, and 14; 3. To decrease suppressor cells at the tumor site, mice can receive i.p. 120 mg/kg Gem to target myeloid-derived suppressor cells (MDSC). There is clinical and experimental evidence that cancer tissues with high infiltration of MDSCs are associated with poor patient prognosis and resistance to therapies. Our experiments showed that blockade of MDSC cells improved ACT-TIL therapy in melanoma and we have measured high numbers of MDSC in MB49 and patient bladder tumors. Tumor measurement can be recorded 2-3 per week. In additional experiments, tumors can be collected at various time points after TIL delivery (1, 3, 7 and 14 days and at endpoint) for IHC, flow cytometric and functional assays as described herein.

### e) Validate Optimal Treatment Protocols in the Murine Model of Bladder Cancer.

Model predictions can be tested in orthotopic MB49 model using schedules and doses determined by in silico model that can provide disparate outcomes in terms of tumor responses. This protocol can involve four treatment cohorts: (a) vehicle control; (b) a test dose determined in silico that results in maximal tumor control; (c) a test dose determined in silico that results in tumor control with minimal accumulated dose; and (d) a test dose determined in silico that results in tumor control with minimal number of therapeutic interventions. Tumor burdens can be quantified weekly by ultrasound. Differences between predicted and actual tumor growth inhibition (TGI) can be analyzed by Bland-Altman statistics. For murine models, male and female mice can be randomly allocated to experimental groups at age 6 weeks. A group size of n=10 (5 males, 5 females) per treatment cohort can provide at least 80% power to detect statistical differences between treated and control groups with a 5% significance level. The treatment assignment can be blinded to investigators who participate in endpoint analyses. For comparison of treatment strategies in vivo, a one-way ANOVA (followed by Tukey post hoc test) can be performed using tumor measurement taken at each time point. The log-rank test can be used to compare the survival distribution between groups. A Mann-Whitney test (unpaired) or a paired t-test can be used to compare between two treatment groups. Statistical significance can be achieved when p<0.05.

### f) Provide a VirTuOSo Module for Testing Treatment Schedules.

The VirTuOSo module for this can allow for determining the optimal treatment protocols, that is, the order, timing, dosage, treatment duration, and the length of vacation periods (if any) for combination therapies. Thus, this module can include: (i) input data of a time series of tumor sizes from in vivo experiments without treatment and with each mono-therapy; (ii) progressive data fitting for defining the parameters of mathematical cell population models; (iii) simulations of virtual treatment protocols; (iv) implementation of MABS algorithms for optimal protocols determination.

### 5. Example 5: Reconstructing the Oxygenation Landscape of Bladder Tumors in Mice

The tortuous tumor vasculature can cause heterogeneities in tissue oxygenation resulting in well-oxygenated (normoxia) areas and regions with low oxygen (hypoxia) within a tissue. We developed an in silico hybrid agent based model that uses digitized tumor histology images from twelve bladder tumors grown in mice as the base for simulations. This model is used to examine the oxygenation patterns and to investigate the cellular composition of hypoxic versus normoxic regions.

### a) Tissue Design

All cell and vessel coordinates and sizes were determined from tissue histology images following the described pipelines.

### b) Oxygen Kinetics

The change in oxygen concentration y(x,t) at location x at time t depends on its influx I_{γ} from vessels, diffusion through the tissue with a constant diffusion coefficient D_{y}, and uptake by the cells yup (modelled using Michaelis-Menten kinetics to allow for oxygen consumption at different rates depending on the amount of available oxygen).

$$\frac{\partial γ ( x , t )}{\partial t} = {D}_{γ} Δ γ ( x , t ) - {γ}_{up} ( x , t ) {\sum }_{k = 1}^{{N}_{C}} {𝒳}_{{R}_{C}} ( {X}_{k} ( t ) , x )$$

$${𝒳}_{{R}_{C}} ( {X}_{k} ( t ) , x ) = \{ \begin{matrix}1 , & if ‖ x - {X}_{k} ( t ) ‖ ≪ max ( {R}_{C} , hg ) \\ 0 , & otherwise ,\end{matrix}$$

where hg is the grid size, R_{c} is the cell radius, N_{c} is the total number of cells, x=(x,y) are grid coordinates, Xₖ=(X,Y) denotes cell coordinates, and X is the indicator function defining the local neighborhood around Xₖ.

### c) Oxygen and Spatial Distribution

A stable oxygen distribution was generated for the above equation with stability conditions:

$${ε}_{γ}^{n} = {‖ {γ}^{n} - {γ}^{n - 1} ‖}_{2} = \sqrt{{\sum }_{i , j = 1}^{{N}_{i} , {N}_{j}} {( {γ}^{n} - {γ}^{n - 1} )}^{2}} < {10}^{- 5} and \frac{{D}_{γ} Δ t}{{( hg )}^{2}} \leq \frac{1}{4}$$

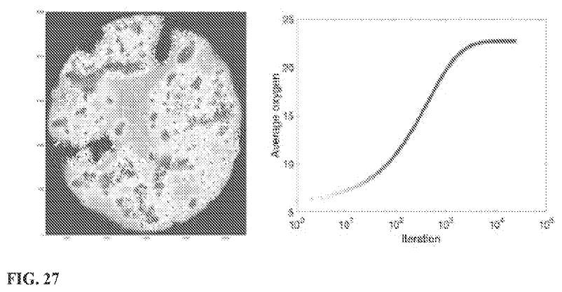

As shown in the left panel of FIG. 27 we observe a stable oxygen distribution. On the right panel we show the average oxygen steadily increases and reaches equilibrium at ˜22 mmHg.

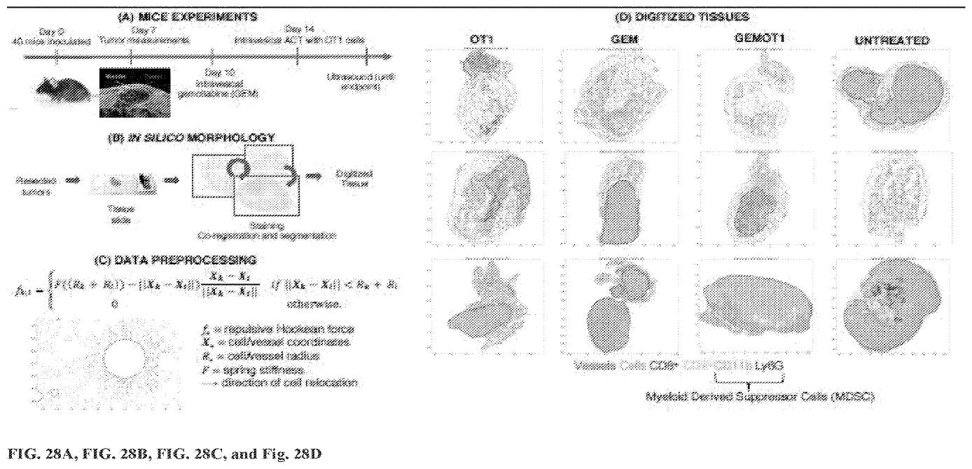

Next we looked at a model in using in vivo and in silico measures. As shown in FIG. 28A, immunocompetent mice were injected with MB-OVA tumor cells then treated with gemcitabine and/or adoptive cell therapy with OT1 cells. FIG. 28B shows that resected tumors were sliced and stained for cells (H&E), vasculature (CD31) and immune cells (CD4+, CD8+, CD11b, Ly6G), then scanned and digitized. FIG. 28C shows that repulsive were calculated for cell-to-cell and cell-to-vessel interactions to avoid overlapping. FIG. 28D shows the 12 digitized tissues. The black lines inside some tissues enclose the tumor regions.

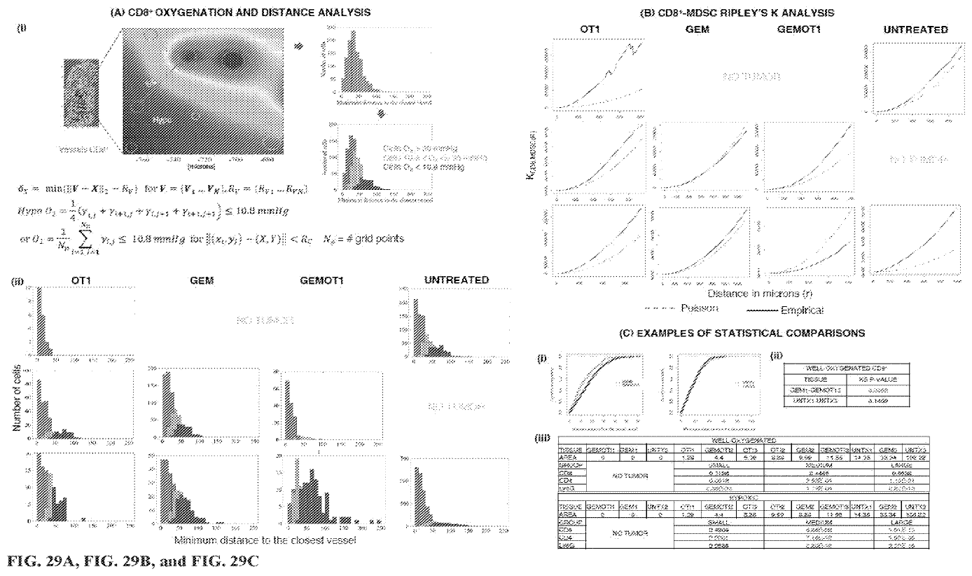

We then performed an analysis of the cells' oxygenation and clustering patterns in the tumor regions. FIG. 29A shows in panel (i) A portion of GEM tissue showing the immune cells in different oxygenated regions. FIG. 29A in panel (ii) shows histograms of the oxygenation level and minimum distances of CD8+ cells from the closest vessel. FIG. 29B shows the Ripley's K analysis of CD8+ and MDSCs cells across the treatments (solid line above clumped, below dispersed). FIG. 29C shows in panel (i) empirical cumulative distribution functions for different (left) and similar (right) distributions. FIG. 29C in panel (ii) shows Kolgomorov-Smirvov pairwise comparison. FIG. 29C in panel (iii) shows Kruskal-Wallis p-values for comparing distributions across the treatments grouped by tumor sizes (black-similar, red-different) for the well-oxygenated and hypoxic cells.

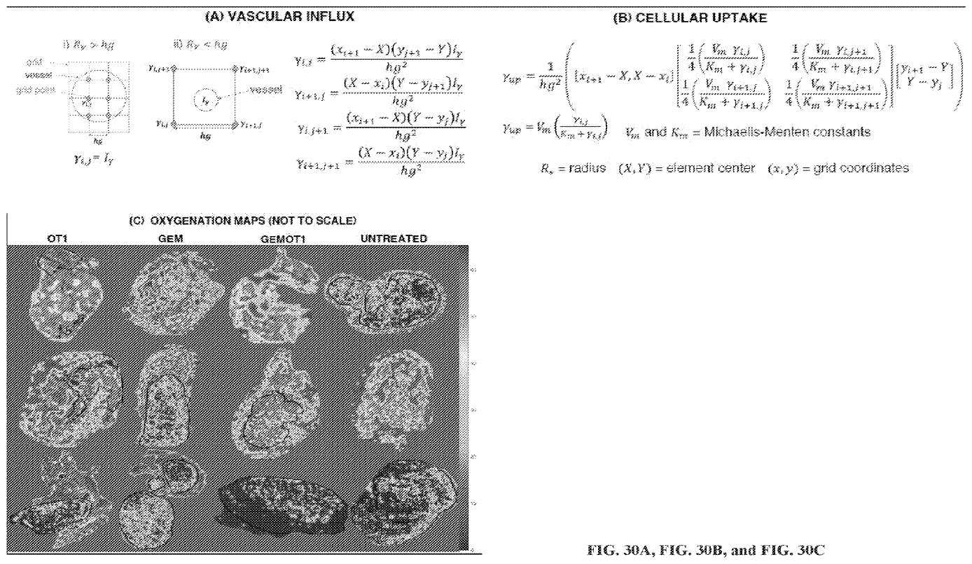

Next we simulated oxygen landscapes. FIG. 30A shows a model of the vascular influx as a boundary condition. FIG. 30A in panel (i) shows the pO2 in a vessel is constant. FIG. 30A in panel (ii) the pO2 at each grid point surrounding the vessel is inversely proportional to the distance from the vessel center. FIG. 30B shows the cellular uptake is modelled in a similar manner. FIG. 30C shows the numerically stable oxygen maps. The smaller tissues (first and second rows) are well oxygenated and have fewer hypoxic regions compared to the larger tissues (last row).

We showed here that histology images with in-house algorithms of cell segmentation reconstructed tissue oxygenation and determined distributions of oxygenated cells. We also showed that a stable oxygenation map was achieved for all simulations based on diffusion-reaction equations. We also observed that the smaller tissues were well vascularized and thus were well-oxygenated compared to the large tissues which had more hypoxic immune cells in the tumor regions. Lastly, we found that tissues treated with GEMOT1 and OT1 had more well-oxygenated CD8+ and MDSCs cells compared to GEM and untreated tissues.

### 6. Example 6: Reconstructing the Metabolic Landscape from Histology Images of Solid Cancers

The tortuous tumor vasculature and irregular cellular architecture can modify the metabolic landscape resulting in heterogeneities in tissue oxygenation. To examine the distribution of intratumoral metabolites, we developed an in silico hybrid agent-based model with Michaelis-Menten kinetics for oxygen uptake and a constant influx of oxygen from the vasculature. This model uses digitized tumor histology images from pancreatic and bladder cancers as the base for simulations.

### a) Tissue Design

All cell Xᵢ and vessel Vⱼ coordinates and sizes were determined from tissue histology images following the described pipelines.

### b) Oxygen Kinetics

The change in oxygen concentration y(x,t) at location x=(x,y) at time t depends on its influx I_{γ} from vessels Vⱼ, diffusion through the tissue with a constant diffusion coefficient D_{y}, and uptake by the cells α_{γ} which is modelled using Michaelis-Menten kinetics to allow for oxygen consumption at different rates depending on the amount of available oxygen.

$$\frac{\partial γ ( x , t )}{\partial t} = {D}_{γ} Δ γ ( x , t ) - α ( γ ) {\sum }_{k = 1}^{{N}_{C}} {𝒳}_{{R}_{C}} ( {X}_{k} ( t ) , x ) + {I}_{γ} {\sum }_{j = 1}^{{N}_{V}} {𝒳}_{{R}_{V}} ( {V}_{j} , x )$$

$$α ( γ ) = {V}_{m} ( \frac{γ ( x , t )}{γ ( x , t ) + {K}_{m}} )$$

$${𝒳}_{R} * ( Z * ( t ) , x ) = \{ \begin{matrix}1 , & if ‖ x - Z * ( t ) ‖ < R * \\ 0 , & otherwise .\end{matrix}$$

Where Vₘ is the maximum oxygen consumption rate, Kₘ is the oxygen concentration at which the uptake rate is one half of the max, R* is either Rᵥ or R_{c}, and X*(t) is Xₖ(t) or Vⱼ.

### a) Oxygen and Spatial Distribution

A stable oxygen distribution was generated for the above equation with stability condition:

$$\frac{{D}_{γ} Δ t}{{( hg )}^{2}} \leq \frac{1}{4}$$

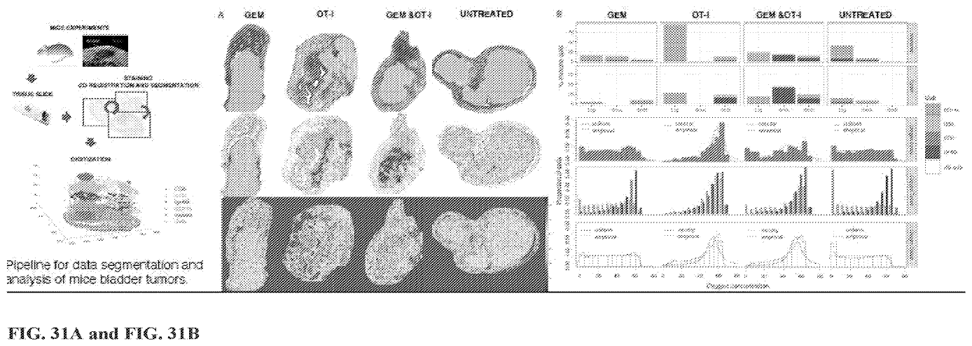

As shown in the left panel of FIG. 27 we observe a stable oxygen distribution. On the right panel we show the average oxygen steadily increases and reaches equilibrium at ˜22 mmHg. We used our model to observe the immune landscape in bladder cancer after treatment (FIGS. 31A and 31B). FIG. 31A shows histology from mouse bladder tumors: untreated, and treated with gemcitabine (GEM), adoptive T cell therapy (OT-I), and combination of GEM and OT-I, were segmented into tumor and nontumor regions (top row), digitized into immune and tumor cells (middle row), and used to simulate the stable oxygen distribution (bottom row). FIG. 31B shows the immune cell proportions varied between the large (Lrg) and small (SmA and SmB) bladder tissues, and across tumor and nontumor regions. The histograms and empirical distributions of oxygenated CD8+, CD11b+, Ly6G+, and all cells showed different distributions for untreated tumor and the treated tumors.

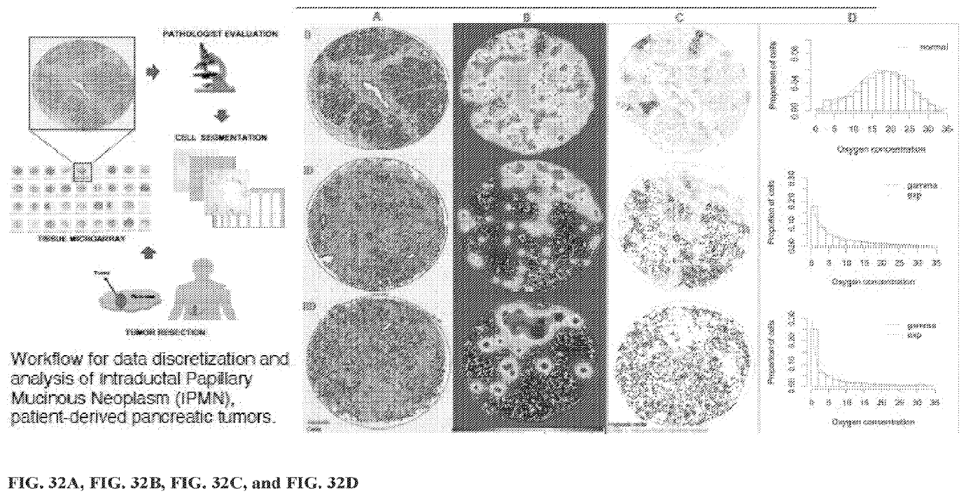

Next we measured the oxygen landscape of IPMN tumors. Pancreas tumors of different grades i) benign ii) premalignant with fibrotic stroma, iii) invasive with desmoplastic stroma, were discretized (32A) and used to simulate the stable oxygen distribution (32B). The hypoxic cells were identified (32C). FIG. 32D shows the histograms and empirical distributions of oxygenated vs. hypoxic tumor cells showed different distributions of: benign tissue-Normal (17.72,6.82), pre-malignant tumor-Gamma (0.71, 0.11), and invasive tumor-Gamma (0.4,0.072).

We show here that Histology images with in-house algorithms of cell segmentation reconstructed tissue oxygenation and determined distributions of oxygenated cells. The stable oxygenation map was achieved for all simulations based on diffusion-reaction equations. All bladder tissues were well vascularized and thus were well-oxygenated, and most of the immune cells were in normoxic regions. We also showed that the benign pancreatic tissue had a normal oxygenation pattern while the premalignant and invasive tumors did not. Finally, we show that the cell-scale simulations correlated the oxygen distribution patterns with 1) pancreatic tumor grades and 2) bladder tumor-T cell infiltration potential.

### D. References

Anders S, Pyl P T, Huber W. HTSeq—a Python framework to work with high-throughput sequencing data. Bioinformatics (Oxford, England). 2015; 31 (2): 166-9. Epub 2014 Sep. 28. doi: 10.1093/bioinformatics/btu638. PubMed PMID: 25260700; PMCID: PMC4287950.
Arora J S. Introduction to Optimum Design: Elsevier; 2017.
Audet C, Dennis J E. Mesh adaptive direct search algorithms for constrained optimization. SIAM Journal on Optimization. 2006; 17 (1): 188-217.
Aydin A M, Bunch B L, Beatty M, Hajiran A, Dhillon J, Sarnaik A A, Pilon-Thomas S, Poch M A. The Factors Affecting Expansion of Reactive Tumor Infiltrating Lymphocytes (TIL) From Bladder Cancer and Potential Therapeutic Applications. Front Immunol. 2021; 12:628063. Epub 2021 Mar. 16. doi: 10.3389/fimmu.2021.628063. PubMed PMID: 33717150; PMCID: PMC7949015.
Brown D W. Smoking prevalence among US veterans. J Gen Intern Med. 2010; 25 (2): 147-9. doi: 10.1007/s11606-009-1160-0. PubMed PMID: 19894079; PMCID: PMC2837499.
Cancer Genome Atlas Research N. Comprehensive molecular characterization of urothelial bladder carcinoma. Nature. 2014; 507 (7492): 315-22. doi: 10.1038/nature12965. PubMed PMID: 24476821; PMCID: PMC3962515.
Chacon J A, Pilon-Thomas S, Sarnaik A A, Radvanyi L G. Continuous 4-1BB co-stimulatory signals for the optimal expansion of tumor-infiltrating lymphocytes for adoptive T-cell therapy. Oncoimmunology. 2013; 2 (9): e25581. doi: 10.4161/onci.25581. PubMed PMID: 24319633; PMCID: PMC3850170.
Chacon J A, Sarnaik A A, Chen J Q, Creasy C, Kale C, Robinson J, Weber J, Hwu P, Pilon-Thomas S, Radvanyi L. Manipulating the tumor microenvironment ex vivo for enhanced expansion of tumor-infiltrating lymphocytes for adoptive cell therapy. Clinical cancer research: an official journal of the American Association for Cancer Research. 2015; 21 (3): 611-21. doi: 10.1158/1078-0432.CCR-14-1934. PubMed PMID: 25472998; PMCID: PMC4315752.
Chen F, Zhang G, Cao Y, Hessner M J, See W A. MB49 murine urothelial carcinoma: molecular and phenotypic comparison to human cell lines as a model of the direct tumor response to bacillus Calmette-Guerin. J Urol. 2009; 182 (6): 2932-7. doi: 10.1016/j.juro.2009.08.018. PubMed PMID: 19853870.
Chikh G, de Jong S D, Sekirov L, Raney S G, Kazem M, Wilson K D, Cullis P R, Dutz J P, Tam Y K. Synthetic methylated CpG ODNs are potent in vivo adjuvants when delivered in liposomal nanoparticles. Int Immunol. 2009; 21 (7): 757-67. doi: 10.1093/intimm/dxp044. PubMed PMID: 19502586.
de Jong S D, Basha G, Wilson K D, Kazem M, Cullis P, Jefferies W, Tam Y. The immunostimulatory activity of unmethylated and methylated CpG oligodeoxynucleotide is dependent on their ability to colocalize with TLR9 in late endosomes. J Immunol. 2010; 184 (11): 6092-102. doi: 10.4049/jimmunol.0802442. PubMed PMID: 20427776.
DePristo M A, Banks E, Poplin R, Garimella K V, Maguire J R, Hartl C, Philippakis A A, del Angel G, Rivas M A, Hanna M, McKenna A, Fennell T J, Kernytsky A M, Sivachenko A Y, Cibulskis K, Gabriel S B, Altshuler D, Daly M J. A framework for variation discovery and genotyping using next-generation DNA sequencing data. Nature genetics. 2011; 43 (5): 491-8. doi: 10.1038/ng.806. PubMed PMID: 21478889; PMCID: PMC3083463.
Eisenberg M C, Jain H V. A confidence building exercise in data and identifiability: Modeling cancer chemotherapy as a case study. J Theor Biol. 2017; 431:63-78. doi: 10.1016/j.jtbi.2017.07.018. PubMed PMID: 28733187; PMCID: PMC6007023.
Gabrilovich D I, Ostrand-Rosenberg S, Bronte V. Coordinated regulation of myeloid cells by tumours. Nat Rev Immunol. 2012; 12 (4): 253-68. doi: 10.1038/nri3175. PubMed PMID: 22437938; PMCID: PMC3587148.
Ghansah T, Vohra N, Kinney K, Weber A, Kodumudi K, Springett G, Sarnaik A A, Pilon-Thomas S. Dendritic cell immunotherapy combined with gemcitabine chemotherapy enhances survival in a murine model of pancreatic carcinoma. Cancer Immunol Immunother. 2013; 62 (6): 1083-91. doi: 10.1007/s00262-013-1407-9. PubMed PMID: 23604104; PMCID: PMC3666559.
Gommes C J, Jiao Y, Torquato S. Density of States for a specified correlation function and the energy landscape. Physical review letters. 2012; 108 (8): 080601. Epub 2012 Apr. 3. doi: 10.1103/PhysRevLett.108.080601. PubMed PMID: 22463509.
Gopalakrishnan S, Majumder K, Predeus A, Huang Y, Koues O I, Verma-Gaur J, Loguercio S, Su A I, Feeney A J, Artyomov M N, Oltz E M. Unifying model for molecular determinants of the preselection Vbeta repertoire. Proceedings of the National Academy of Sciences of the United States of America. 2013; 110 (34): E3206-15. Epub 2013 Aug. 7. doi: 10.1073/pnas. 1304048110. PubMed PMID: 23918392; PMCID: 3752219.
Grasso M, Torelli F, Scannapieco G, Franzoso F, Lania C. Neoadiuvant treatment with intravesical interleukin-2 for recurrent superficial transitional bladder carcinoma Ta-T1/G1-2. Journal of immunotherapy (Hagerstown, Md: 1997). 2001; 24 (2): 184-7. Epub 2001 Mar. 27. PubMed PMID: 11265776.
Gropper Y, Feferman T, Shalit T, Salame T M, Porat Z, Shakhar G. Culturing CTLs under Hypoxic Conditions Enhances Their Cytolysis and Improves Their Anti-tumor Function. Cell Rep. 2017; 20 (11): 2547-55. doi: 10.1016/j.celrep.2017.08.071. PubMed PMID: 28903036.
Haas G P, Solomon D, Rosenberg S A. Tumor-infiltrating lymphocytes from nonrenal urological malignancies. Cancer immunology, immunotherapy: CII. 1990; 30 (6): 342-50. Epub 1990 Jan. 1. PubMed PMID: 2105845.
Hartana C A, Ahlen Bergman E, Broome A, Berglund S, Johansson M, Alamdari F, Jakubczyk T, Huge Y, Aljabery F, Palmqvist K, Holmstrom B, Glise H, Riklund K, Sherif A, Winqvist O. Tissue-resident memory T cells are epigenetically cytotoxic with signs of exhaustion in human urinary bladder cancer. Clinical and experimental immunology. 2018. doi: 10.1111/cei.13183. PubMed PMID: 30009527.
Housseau F, Zeliszewski D, Roy M, Paradis V, Richon S, Ricour A, Bougaran J, Prapotnich D, Vallancien G, Benoit G, Desportes L, Bedossa P, Hercend T, Bidart J M, Bellet D. MHC-dependent cytolysis of autologous tumor cells by lymphocytes infiltrating urothelial carcinomas. International journal of cancer Journal international du cancer. 1997; 71 (4): 585-94. Epub 1997 May 16. PubMed PMID: 9178812.
Huebner D, Rieger C, Bergmann R, Ullrich M, Meister S, Toma M, Wiedemuth R, Temme A, Novotny V, Wirth M P, Bachmann M, Pietzsch J, Fuessel S. An orthotopic xenograft model for high-risk non-muscle invasive bladder cancer in mice: influence of mouse strain, tumor cell count, dwell time and bladder pretreatment. BMC Cancer. 2017; 17 (1): 790. doi: 10.1186/s12885-017-3778-3. PubMed PMID: 29169339; PMCID: PMC5701455.
Hypoxia in cancer chemo- and immunotherapy: foe or friend? [Internet]. Cold Spring Harbor Laboratory. 2019
Kamat A M, Briggman J, Urbauer D L, Svatek R, Nogueras Gonzalez G M, Anderson R, Grossman H B, Prat F, Dinney C P. Cytokine Panel for Response to Intravesical Therapy (CyPRIT): Nomogram of Changes in Urinary Cytokine Levels Predicts Patient Response to Bacillus Calmette-Guerin. Eur Urol. 2016; 69 (2): 197-200. doi: 10.1016/j.eururo.2015.06.023. PubMed PMID: 26119560; PMCID: PMC4691211.
Karolak A, Agrawal S, Lee S, Rejniak K A. Single-Cell-Based In Silico Models: A Tool for Dissecting Tumor Heterogeneity. In: Narayan S, editor. Encyclopedia of Biomedical Engineering: Elsevier; 2018.
Karolak A, Agrawal S, Lee S, Rejniak K A. Single-Cell-Based in Silico Models: A Tool for Dissecting Tumor Heterogeneity In: Narayan S, editor. Encyclopedia of Biomedical Engineering: Elsevier; 2019. p. 130-43.
Karolak A, Estrella V C, Huynh A S, Chen T, Vagner J, Morse D L, Rejniak K A. Targeting Ligand Specificity Linked to Tumor Tissue Topological Heterogeneity via Single-Cell Micro-Pharmacological Modeling. Sci Rep. 2018; 8 (1): 3638. doi: 10.1038/s41598-018-21883-z. PubMed PMID: 29483578; PMCID: PMC5827036.
Karolak A, Markov D A, McCawley L J, Rejniak K A. Towards personalized computational oncology: from spatial models of tumour spheroids, to organoids, to tissues. J R Soc Interface. 2018; 15 (138). Epub 2018 Jan. 26. doi: 10.1098/rsif.2017.0703. PubMed PMID: 29367239; PMCID: PMC5805971.
Karolak A, Poonja S, Rejniak K A. Morphophenotypic classification of tumor organoids as an indicator of drug exposure and penetration potential. PLOS Comput Biol. 2019; 15 (7): e1007214. Epub 2019 Jul. 17. doi: 10.1371/journal.pcbi.1007214. PubMed PMID: 31310602; PMCID: PMC6660094.
Karolak A, Rejniak K A. Micropharmacology: An In Silico Approach for Assessing Drug Efficacy Within a Tumor Tissue. Bull Math Biol. 2019; 81 (9): 3623-41. Epub 2018 Feb. 10. doi: 10.1007/s11538-018-0402-x. PubMed PMID: 29423880; PMCID: PMC6082744.
Kim M, Gillies R J, Rejniak K A. Current advances in mathematical modeling of anti-cancer drug penetration into tumor tissues. Front Oncol. 2013; 3:278. Epub 2013 Dec. 5. doi: 10.3389/fonc.2013.00278. PubMed PMID: 24303366; PMCID: PMC3831268.
Kingsley J L, Costello J R, Raghunand N, Rejniak K A. Bridging cell-scale simulations and radiologic images to explain short-time intratumoral oxygen fluctuations. PLOS Comput Biol. 2021; 17 (7): e1009206. Epub 2021 Jul. 27. doi: 10.1371/journal.pcbi.1009206. PubMed PMID: 34310608; PMCID: PMC8341701.
Kodumudi K N, Siegel J, Weber A M, Scott E, Sarnaik A A, Pilon-Thomas S. Immune Checkpoint Blockade to Improve Tumor Infiltrating Lymphocytes for Adoptive Cell Therapy. PLOS One. 2016; 11 (4): e0153053. doi: 10.1371/journal.pone.0153053. PubMed PMID: 27050669; PMCID: PMC4822778.
Kodumudi K N, Weber A, Sarnaik A A, Pilon-Thomas S. Blockade of myeloid-derived suppressor cells after induction of lymphopenia improves adoptive T cell therapy in a murine model of melanoma. J Immunol. 2012; 189 (11): 5147-54. doi: 10.4049/jimmunol. 1200274. PubMed PMID: 23100512; PMCID: PMC3505990.
Koike N, Pilon-Thomas S, Mule J J. Nonmyeloablative chemotherapy followed by T-cell adoptive transfer and dendritic cell-based vaccination results in rejection of established melanoma. J Immunother. 2008; 31 (4): 402-12. doi: 10.1097/CJI.0b013e31816cabbb. PubMed PMID: 18391755.
Lardner A. The effects of extracellular pH on immune function. Journal of leukocyte biology. 2001; 69 (4): 522-30. PubMed PMID: 11310837.
Li H, Durbin R. Fast and accurate short read alignment with Burrows-Wheeler transform. Bioinformatics. 2009; 25 (14): 1754-60. doi: 10.1093/bioinformatics/btp324. PubMed PMID: 19451168; PMCID: PMC2705234.
Lloyd M C, Rejniak K A, Brown J S, Gatenby R A, Minor E S, Bui M M. Pathology to enhance precision medicine in oncology: lessons from landscape ecology. Adv Anat Pathol. 2015; 22 (4): 267-72. doi: 10.1097/PAP.0000000000000078. PubMed PMID: 26050264; PMCID: PMC4729443.
Love M I, Huber W, Anders S. Moderated estimation of fold change and dispersion for RNA-seq data with DESeq2. Genome Biol. 2014; 15 (12): 550. doi: 10.1186/s13059-014-0550-8. PubMed PMID: 25516281; PMCID: PMC4302049.
Mahfouz M, Hashimoto W, Das Gupta T K, Chakrabarty A M. Bacterial proteins and CpG-rich extrachromosomal DNA in potential cancer therapy. Plasmid. 2007; 57 (1): 4-17. doi: 10.1016/j.plasmid.2006.11.001. PubMed PMID: 17166586.
Meshkat N, Sullivant S, Eisenberg M. Identifiability Results for Several Classes of Linear Compartment Models. Bull Math Biol. 2015; 77 (8): 1620-51. doi: 10.1007/s11538-015-0098-0. PubMed PMID: 26337290.
Modeling vaccine-induced immunotherapy: treatment scheduling and robustness with virtual mice cohorts [Internet]. Cold Spring Harbor Laboratory. 2019.
Morales A, Eidinger D, Bruce A W. Intracavitary Bacillus Calmette-Guerin in the treatment of superficial bladder tumors. The Journal of urology. 1976; 116 (2): 180-3. Epub 1976 Aug. 1. PubMed PMID: 820877.
Nagaraj S, Schrum A G, Cho H I, Celis E, Gabrilovich D I. Mechanism of T cell tolerance induced by myeloid-derived suppressor cells. J Immunol. 2010; 184 (6): 3106-16. Epub 2010 Feb. 10. doi: 10.4049/jimmunol.0902661. PubMed PMID: 20142361; PMCID: 2832724.
Oyeka I C A, Ebuh G U. Modified Wilcoxon Signed-Rank Test. Open Journal of Statistics. 2012; 2 (2): 172-6.
Parekh D J, Bochner B H, Dalbagni G. Superficial and muscle-invasive bladder cancer: principles of management for outcomes assessments. Journal of clinical oncology: official journal of the American Society of Clinical Oncology. 2006; 24 (35): 5519-27. Epub 2006 Dec. 13. doi: 10.1200/JCO.2006.08.5431. PubMed PMID: 17158537.
Perez-Velazquez J, Rejniak K A. Drug-Induced Resistance in Micrometastases: Analysis of Spatio-Temporal Cell Lineages. Front Physiol. 2020; 11:319. Epub 2020 May 5. doi: 10.3389/fphys.2020.00319. PubMed PMID: 32362836; PMCID: PMC7180185.
Pettenati C, Ingersoll M A. Mechanisms of BCG immunotherapy and its outlook for bladder cancer. Nat Rev Urol. 2018. doi: 10.1038/s41585-018-0055-4. PubMed PMID: 29991725.
Pichler R, Fritz J, Zavadil C, Schafer G, Culig Z, Brunner A. Tumor-infiltrating immune cell subpopulations influence the oncologic outcome after intravesical Bacillus Calmette-Guerin therapy in bladder cancer. Oncotarget. 2016; 7 (26): 39916-30. doi: 10.18632/oncotarget.9537. PubMed PMID: 27221038; PMCID: PMC5129981.
Pilon-Thomas S, Kodumundi K, Luddy K, El-Kenawi A, Weber A, Damaghi M, Wojtkowiak J W, Ibrahim-Hashim A, Mule J, Gillies J. Neutralization of tumor acidity promotes anti-tumor immune therapy. Cancer Research. 2016; 76 (6): 1381-90.
Pilon-Thomas S, Kuhn L, Ellwanger S, Janssen W, Royster E, Marzban S, Kudchadkar R, Zager J, Gibney G, Sondak V K, Weber J, Mule J J, Sarnaik A A. Efficacy of adoptive cell transfer of tumor-infiltrating lymphocytes after lymphopenia induction for metastatic melanoma. J Immunother. 2012; 35 (8): 615-20. Epub 2012 Sep. 22. doi: 10.1097/CJI.0b013e31826e8f5f. PubMed PMID: 22996367.
Pilon-Thomas S, Li W, Briggs J J, Djeu J, Mule J J, Riker A I. Immunostimulatory effects of CpG-ODN upon dendritic cell-based immunotherapy in a murine melanoma model. J Immunother. 2006; 29 (4): 381-7. Epub 2006 Jun. 27. doi: 10.1097/01.cji.0000199199.20717.67. PubMed PMID: 16799333.
Pilon-Thomas S, Mackay A, Vohra N, Mule J J. Blockade of programmed death ligand 1 enhances the therapeutic efficacy of combination immunotherapy against melanoma. J Immunol. 2010; 184 (7): 3442-9. Epub 2010 Mar. 3. doi: 10.4049/jimmunol.0904114. PubMed PMID: 20194714; PMCID: 2913584.
Plimack E R, Bellmunt J, Gupta S, Berger R, Chow L Q, Juco J, Lunceford J, Saraf S, Perini R F, O'Donnell P H. Safety and activity of pembrolizumab in patients with locally advanced or metastatic urothelial cancer (KEYNOTE-012): a non-randomised, open-label, phase 1b study. The lancet oncology. 2017; 18 (2): 212-20. Epub 2017 Jan. 14. doi: 10.1016/S1470-2045 (17) 30007-4. PubMed PMID: 28081914.
Poch M, Hall M, Joerger A, Kodumudi K, Beatty M, Innamarato P P, Bunch B L, Fishman M N, Zhang J, Sexton W J, Pow-Sang J M, Gilbert S M, Spiess P E, Dhillon J, Kelley L, Mullinax J, Sarnaik A A, Pilon-Thomas S. Expansion of tumor infiltrating lymphocytes (TIL) from bladder cancer. Oncoimmunology. 2018; 7 (9): e1476816. doi: 10.1080/2162402X.2018.1476816. PubMed PMID: 30228944; PMCID: PMC6140546.
Powles T, Eder J P, Fine G D, Braiteh F S, Loriot Y, Cruz C, Bellmunt J, Burris H A, Petrylak D P, Teng S L, Shen X, Boyd Z, Hegde P S, Chen D S, Vogelzang N J. MPDL3280A (anti-PD-L1) treatment leads to clinical activity in metastatic bladder cancer. Nature. 2014; 515 (7528): 558-62. Epub 2014 Nov. 28. doi: 10.1038/nature13904. PubMed PMID: 25428503.
Ramiya V K, Jerald M M, Lawman P D, Lawman M J. Autologous tumor cells engineered to express bacterial antigens. Methods Mol Biol. 2014; 1139:243-57. doi: 10.1007/978-1-4939-0345-0_21. PubMed PMID: 24619685.
Rejniak K A, Estrella V, Chen T, Cohen A S, Lloyd M C, Morse D L. The role of tumor tissue architecture in treatment penetration and efficacy: an integrative study. Front Oncol. 2013; 3:111. Epub 2013 May 30. doi: 10.3389/fonc.2013.00111. PubMed PMID: 23717812; PMCID: PMC3650652.
Rejniak K A, Lloyd M C, Reed D R, Bui M M. Diagnostic assessment of osteosarcoma chemoresistance based on Virtual Clinical Trials. Med Hypotheses. 2015; 85 (3): 348-54. doi: 10.1016/j.mehy.2015.06.015. PubMed PMID: 26130106; PMCID: PMC4549200.
Rejniak K A, McCawley L J. Current trends in mathematical modeling of tumor-microenvironment interactions: a survey of tools and applications. Experimental biology and medicine (Maywood, NJ). 2010; 235 (4): 411-23. Epub 2010 Apr. 22. doi: 10.1258/ebm.2009.009230. PubMed PMID: 20407073.
Rejniak K A, McCawley L J. Current trends in mathematical modeling of tumor-microenvironment interactions: a survey of tools and applications. Exp Biol Med (Maywood). 2010; 235 (4): 411-23. Epub 2010 Apr. 22. doi: 10.1258/ebm.2009.009230. PubMed PMID: 20407073.
Rejniak K A, Quaranta V, Anderson A R. Computational investigation of intrinsic and extrinsic mechanisms underlying the formation of carcinoma. Math Med Biol. 2012; 29 (1): 67-84. doi: 10.1093/imammb/dqq021. PubMed PMID: 21106672; PMCID: PMC3499074.
Rosenberg S A, Restifo N P. Adoptive cell transfer as personalized immunotherapy for human cancer. Science. 2015; 348 (6230): 62-8. doi: 10.1126/science.aaa4967. PubMed PMID: 25838374; PMCID: PMC6295668.
Rosenberg S A, Yang J C, Sherry R M, Kammula U S, Hughes M S, Phan G Q, Citrin D E, Restifo N P, Robbins P F, Wunderlich J R, Morton K E, Laurencot C M, Steinberg S M, White D E, Dudley M E. Durable complete responses in heavily pretreated patients with metastatic melanoma using T-cell transfer immunotherapy. Clinical cancer research: an official journal of the American Association for Cancer Research. 2011; 17 (13): 4550-7. Epub 2011 Apr. 19. doi: 10.1158/1078-0432.CCR-11-0116. PubMed PMID: 21498393; PMCID: 3131487.
Schumacher T N, Schreiber R D. Neoantigens in cancer immunotherapy. Science. 2015; 348 (6230): 69-74. doi: 10.1126/science.aaa4971. PubMed PMID: 25838375.
Shabsigh A, Korets R, Vora K C, Brooks C M, Cronin A M, Savage C, Raj G, Bochner B H, Dalbagni G, Herr H W, Donat S M. Defining early morbidity of radical cystectomy for patients with bladder cancer using a standardized reporting methodology. Eur Urol. 2009; 55 (1): 164-74. doi: 10.1016/j.eururo.2008.07.031. PubMed PMID: 18675501.
Shah A B, Rejniak K A, Gevertz J L. Limiting the development of anti-cancer drug resistance in a spatial model of micrometastases. Math Biosci Eng. 2016; 13 (6): 1185-206. doi: 10.3934/mbe.2016038. PubMed PMID: 27775375; PMCID: PMC5113823.
Sharma P, Callahan M K, Bono P, Kim J, Spiliopoulou P, Calvo E, Pillai R N, Ott P A, de Braud F, Morse M, Le D T, Jaeger D, Chan E, Harbison C, Lin C S, Tschaika M, Azrilevich A, Rosenberg J E. Nivolumab monotherapy in recurrent metastatic urothelial carcinoma (CheckMate 032): a multicentre, open-label, two-stage, multi-arm, phase 1/2 trial. The Lancet Oncology. 2016; 17 (11): 1590-8. Epub 2016 Oct. 14. doi: 10.1016/S1470-2045 (16) 30496-X. PubMed PMID: 27733243.
Sharma P, Shen Y, Wen S, Yamada S, Jungbluth A A, Gnjatic S, Bajorin D F, Reuter V E, Herr H, Old L J, Sato E. CD8 tumor-infiltrating lymphocytes are predictive of survival in muscle-invasive urothelial carcinoma. Proc Natl Acad Sci USA. 2007; 104 (10): 3967-72. doi: 10.1073/pnas.0611618104. PubMed PMID: 17360461; PMCID: PMC1820692.
Siegel R L, Miller K D, Jemal A. Cancer statistics, 2018. CA Cancer J Clin. 2018; 68 (1): 7-30. doi: 10.3322/caac.21442. PubMed PMID: 29313949.
Siegel R L, Miller K D, Jemal A. Cancer statistics, 2018. CA: a cancer journal for clinicians. 2018; 68 (1): 7-30. doi: 10.3322/caac.21442. PubMed PMID: 29313949.
Sitkovsky M V, Kjaergaard J, Lukashev D, Ohta A. Hypoxia-adenosinergic immunosuppression: tumor protection by T regulatory cells and cancerous tissue hypoxia. Clinical cancer research: an official journal of the American Association for Cancer Research. 2008; 14 (19): 5947-52. doi: 10.1158/1078-0432.CCR-08-0229. PubMed PMID: 18829471.
Stephens M A. EDF Statistics for Goodness of Fit and Some Comparisons. Journal of the American Statistical Association. 1974; 69 (347): 730-7. doi: 10.1080/01621459.1974.10480196.
Stevanović S, Draper L M, Langhan M M, Campbell T E, Kwong M L, Wunderlich J R, Dudley M E, Yang J C, Sherry R M, Kammula U S, Restifo N P, Rosenberg S A, Hinrichs C S. Complete regression of metastatic cervical cancer after treatment with human papillomavirus-targeted tumor-infiltrating T cells. Journal of clinical oncology: official journal of the American Society of Clinical Oncology. 2015; 33 (14): 1543-50. Epub 2015 Apr. 1. doi: 10.1200/jco.2014.58.9093. PubMed PMID: 25823737; PMCID: PMC4417725 online at www.jco.org. Author contributions are found at the end of this article.
Sylvester R J, Brausi M A, Kirkels W J, Hoeltl W, Calais Da Silva F, Powell P H, Prescott S, Kirkali Z, van de Beek C, Gorlia T, de Reijke T M, Group E G-UTC. Long-term efficacy results of EORTC genito-urinary group randomized phase 3 study 30911 comparing intravesical instillations of epirubicin, bacillus Calmette-Guerin, and bacillus Calmette-Guerin plus isoniazid in patients with intermediate- and high-risk stage Ta Tl urothelial carcinoma of the bladder. Eur Urol. 2010; 57 (5): 766-73. doi: 10.1016/j.eururo.2009.12.024. PubMed PMID: 20034729; PMCID: PMC2889174
Teer J K, Green E D, Mullikin J C, Biesecker L G. VarSifter: visualizing and analyzing exome-scale sequence variation data on a desktop computer. Bioinformatics. 2012; 28 (4): 599-600. doi: 10.1093/bioinformatics/btr711. PubMed PMID: 22210868; PMCID: PMC3278764.
Teer J K, Zhang Y, Chen L, Welsh E A, Cress W D, Eschrich S A, Berglund A E. Evaluating somatic tumor mutation detection without matched normal samples. Hum Genomics. 2017; 11 (1): 22. doi: 10.1186/s40246-017-0118-2. PubMed PMID: 28870239; PMCID: PMC5584341.
Trapnell C, Pachter L, Salzberg S L. TopHat: discovering splice junctions with RNA-Seq. Bioinformatics. 2009; 25 (9): 1105-11. doi: 10.1093/bioinformatics/btp120. PubMed PMID: 19289445; PMCID: PMC2672628.
Turner M G, Gardner R H. Landscape Ecology in Theory and Proctise. Patterns and Processes: Springer; 2015.
Wang K, Li M, Hakonarson H. ANNOVAR: functional annotation of genetic variants from high-throughput sequencing data. Nucleic Acids Res. 2010; 38 (16): e164. doi: 10.1093/nar/gkq603. PubMed PMID: 20601685; PMCID: PMC2938201.
Wang L, Wang S, Li W. RSeQC: quality control of RNA-seq experiments. Bioinformatics. 2012; 28 (16): 2184-5. doi: 10.1093/bioinformatics/bts356. PubMed PMID: 22743226.
Wojtkowiak J W, Cornnell H C, Matsumoto S, Saito K, Takakusagi Y, Dutta P, Kim M, Zhang X, Leos R, Bailey K M, Martinez G, Lloyd M C, Weber C, Mitchell J B, Lynch R M, Baker A F, Gatenby R A, Rejniak K A, Hart C, Krishna M C, Gillies R J. Pyruvate sensitizes pancreatic tumors to hypoxia-activated prodrug TH-302. Cancer Metab. 2015; 3 (1): 2. doi: 10.1186/s40170-014-0026-z. PubMed PMID: 25635223; PMCID: PMC4310189.
Zaharoff D A, Hoffman B S, Hooper H B, Benjamin C J, Jr., Khurana K K, Hance K W, Rogers C J, Pinto P A, Schlom J, Greiner J W. Intravesical immunotherapy of superficial bladder cancer with chitosan/interleukin-12. Cancer Res. 2009; 69 (15): 6192-9. doi: 10.1158/0008-5472.CAN-09-1114. PubMed PMID: 19638573; PMCID: PMC2788203.
Zullig L L, Sims K J, McNeil R, Williams C D, Jackson G L, Provenzale D, Kelley M J. Cancer Incidence Among Patients of the U.S. Veterans Affairs Health Care System: 2010 Update. Mil Med. 2017; 182 (7): e1883-e91. doi: 10.7205/MILMED-D-16-00371. PubMed PMID: 28810986; PMCID: PMC5650119.

1. A method of treating a cancer in a Bacillus Calmette-Guerin (BCG) unresponsive subject comprising administering to the subject an adoptive cell therapy (ACT).

2. The method of claim 1, wherein the adoptive cell therapy comprises the administration of immune cells selected from the group consisting of tumor infiltrating lymphocytes (TILs), marrow infiltrating lymphocytes (MILs), chimeric antigen receptor (CAR) T cells, CAR macrophage (CARMA), CAR Natural Killer cells (CAR NK cells), and CAR NK T cells.

3. The method of claim 1, further comprising administering to the subject BCG.

4. The method of claim 1, further comprising administering to the subject gemcitabine.

5. The method of claim 4, wherein the immune cells are administered before, concurrent with, simultaneously with, or after administration of gemcitabine.

6. The method of claim 1, wherein the cancer is bladder cancer.

7. The method of claim 6, wherein the bladder cancer is localized non-muscle invasive bladder cancer.

8. The method of claim 2, wherein the immune cells are administered intravesically to the site of the tumor.

9. The method of claim 2, wherein the method does not require preconditioning of the TILs prior to administration.

10. The method of claim 2, wherein the immune cells are TILs.

11. The method of claim 2, wherein the immune cells are expanded ex vivo prior to administration.

12. The method of claim 11, further comprising selecting tumor-reactive immune cells after ex vivo expansion.
